(19) United States
(12) Patent Application Publication
(10) Pub. No.: US 2026/0256723 A1
(43) Pub. Date: Sep. 3, 2026

(54) THIOL-BASED COMPOUNDS AND COMPOSITIONS AND USES THEREOF

(71) Applicant: CASE WESTERN RESERVE UNIVERSITY, Cleveland, OH (US)

(72) Inventors: Stephen J. Lewis, Cleveland Heights, OH (US); Benjamin Gaston, Cleveland, OH (US); James N. Bates, Cleveland, OH (US)

(21) Appl. No.: 18/875,350

(22) PCT Filed: Jun. 16, 2023
(86) PCT No.: PCT/US2023/025563

§ 371 Date: Dec. 16, 2024

Related U.S. Application Data
(60) Provisional application No. 63/352,735, filed on Jun. 16, 2022.
(60) Provisional application No. 63/412,568, filed on Oct. 3, 2022.

(51) Int. Cl. A61K 31/198; A61K 31/16; A61K 31/223; A61P 11/16; A61P 25/36

(52) U.S. Cl. CPC A61K 31/198; A61K 31/16; A61K 31/223; A61P 11/16; A61P 25/36

(57) ABSTRACT

A method of attenuating opioid induced ventilatory and/or respiratory depression and/or reducing opioid induced constipation and/or anuria and/or treating brain injury in a subject in need thereof includes administering to the subject a therapeutically effective amount of a composition comprising a compound having the structure of the formulas (I-VIII).

### RELATED APPLICATION

This application claims priority from U.S. Provisional Application Nos. 63/352,735, filed Jun. 16, 2022 and 63/412,568, filed Oct. 3, 2022, the subject matter of which are incorporated herein by reference in their entirety.

### GOVERNMENT FUNDING

This invention was made with government support under DA051373 awarded by The National Institutes of Health. The government has certain rights in the invention.

### BACKGROUND

The clinical effectiveness of opioid analgesics are often compromised by their adverse effects on breathing. Opioid-induced respiratory depression (OIRD) can in many cases be reversed by opioid receptor antagonists including naloxone but these antagonists also block the analgesic actions of opioids, which may not be problematic in unexpected overdose situations, but which is unwanted when analgesia is required such as during and immediately following surgery. Several classes of non-opioid receptor antagonist agents have been investigated as potential OIRD reversal drugs. The majority of these OIRD reversal agents did not enter into human clinical trials or have successful outcomes in such trials because of lack of efficacy and/or high degrees of toxicity/side effects. Accordingly, there remains an urgent unmet need to introduce drugs that effectively reverse OIRD by mechanisms independent of opioid receptor blockade.

Fentanyl is high-potency opioid receptor (OR) agonist that is widely used to treat both acute and chronic pain. The misuse/abuse of fentanyl and analogues, such as sufentanil and carfentanil, leads to adverse consequences, including often lethal depression of ventilation. Fentanyl is thought of as a selective μ-OR agonist and has very high affinity for μ-ORs. However, fentanyl also activates δ- and κ-ORs with affinities and intrinsic activities of biological significance. For example, whereas fentanyl has low affinity for κ-ORs it has a remarkably high efficacy at these receptors. The mechanisms responsible for the ventilatory depressant and analgesic effects of fentanyl and analogues have been studied extensively. Pre-treatment of rats with naloxone methiodide, a peripherally-restricted —OR antagonist, attenuated fentanyl-induced analgesia, decreases in tidal volume (TV) and increases in Alveolar-arterial (A-a) gradient that were indicative of ventilation-perfusion mismatch or shunting in the lungs. Accordingly, it is likely that the pharmacological actions of fentanyl involve a mixture of effects in the periphery (e.g., vagal cardiopulmonary afferents, the chest-wall and carotid bodies), brain regions, such as the area postrema that are devoid of a blood-brain barrier, and also brain structures within the blood brain barrier, such as the nucleus tractus solitarius.

### SUMMARY

Embodiments described herein relate to thiol-based compounds and their use in pharmaceutical compositions and methods of attenuating opioid induced ventilatory and/or respiratory depression, attenuating opioid induced constipation and/or anuria, and/or treating brain injury in a subject in need thereof.

In some embodiments, thiol-based compounds can have a structure of formulas:

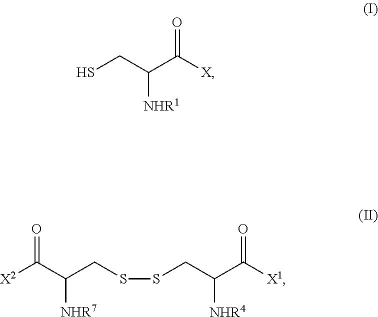

or an adduct, a pharmaceutically acceptable salt, a tautomer, or a solvate thereof; wherein:
X is OR² or N(R³)₂;
X¹ is OR⁵ or N(R⁶)₂;
X² is OR⁸ or N(R⁹)₂;
R¹, R⁴, and R⁷ are each independently H, alkyl optionally substituted with one or more halogen, or —C(O)-alkyl optionally substituted with one or more halogen;
R², R⁵, and R⁸ are each independently H or alkyl optionally substituted with one or more halogen;
each R³, R⁶, and R⁹ are independently H or alkyl optionally substituted with one or more halogen;
X is not OR² if R¹ is H; and
X² is not OR⁸ if X¹ is OR⁵ and R⁴ and R⁷ are H.

In some embodiments, the adduct of the compounds of formulas I or II can be a biologically active adduct and include at least one of an albumin adduct, a glucose adduct, an L-cysteine adduct, L-glutathione adduct, an D-cysteine adduct, or an S-nitroso adduct.

In other embodiments, the compound is not cysteine, cystine, a cysteine alkylester, or cystine dialkylester.

In some embodiments, R¹, R⁴, and R⁷ are each independently H, C₁-C₆ alkyl optionally substituted with one or more halogen, or —C(O)—(C₁-C₆ alkyl) optionally substituted with one or more halogen.

In other embodiments, R¹, R⁴, and R⁷ are each independently H, methyl, ethyl, propyl, butyl, —C(O)-methyl, —C(O)-ethyl, —C(O)-propyl, or —C(O)-butyl, each optionally substituted with one or more halogen.

In some embodiments, R², R⁵, and R⁸ are each independently H or C₁-C₆ alkyl optionally substituted with one or more halogen.

In other embodiments, R², R⁵, and R⁸ are each independently H, methyl, ethyl, or propyl, or butyl, each optionally substituted with one or more halogen.

In some embodiments, each R³, R⁶, and R⁹ is independently H or C₁-C₆ alkyl optionally substituted with one or more halogen.

In other embodiments, one of each R³, R⁶, and R⁹ is H and the other of R³, R⁶, and R⁹ is methyl, ethyl, or propyl, or butyl, each optionally substituted with one or more halogen.

In some embodiments, X is OR²; R¹ is H, C₁-C₆ alkyl optionally substituted with one or more halogen, or —C(O)—(C₁-C₆ alkyl) optionally substituted with one or more halogen; and R² is H or C₁-C₆ alkyl optionally substituted with one or more halogen.

In other embodiments, X is OR²; R¹ is H, methyl, ethyl, propyl, butyl, —C(O)— methyl, —C(O)-ethyl, —C(O)-propyl, or —C(O)-butyl, each optionally substituted with one or more halogen; and R² is H, methyl, ethyl, or propyl, or butyl, each optionally substituted with one or more halogen.

In some embodiments, X is N(R³)₂; R¹ is H, C₁-C₆ alkyl optionally substituted with one or more halogen, or —C(O)—(C₁-C₆ alkyl) optionally substituted with one or more halogen; and each R³ is independently H or C₁-C₆ alkyl optionally substituted with one or more halogen.

In other embodiments, X is N(R³)₂; R¹ is H, methyl, ethyl, propyl, butyl, —C(O)— methyl, —C(O)-ethyl, —C(O)-propyl, or —C(O)-butyl, each optionally substituted with one or more halogen; and one R³ is H and the other R³ is methyl, ethyl, or propyl, or butyl, each optionally substituted with one or more halogen.

In still other embodiments, X¹ is OR⁵; R⁴ is H, C₁-C₆ alkyl optionally substituted with one or more halogen, or —C(O)—(C₁-C₆ alkyl) optionally substituted with one or more halogen; R⁵ is H or C₁-C₆ alkyl optionally substituted with one or more halogen; X² is OR⁸; R⁷ is H, C₁-C₆ alkyl optionally substituted with one or more halogen, or —C(O)—(C₁-C₆alkyl) optionally substituted with one or more halogen; and R⁸ is H or C₁-C₆ alkyl optionally substituted with one or more halogen.

In other embodiments, X¹ is OR⁵; R⁴ is H, methyl, ethyl, propyl, butyl, —C(O)— methyl, —C(O)-ethyl, —C(O)-propyl, or —C(O)-butyl, each optionally substituted with one or more halogen; R⁵ is H, methyl, ethyl, or propyl, or butyl, each optionally substituted with one or more halogen; X² is OR⁸; R⁷ is H, methyl, ethyl, propyl, butyl, —C(O)-methyl, —C(O)-ethyl, —C(O)-propyl, or —C(O)-butyl, each optionally substituted with one or more halogen; and R⁸ is H, methyl, ethyl, or propyl, or butyl, each optionally substituted with one or more halogen.

In some embodiments, X¹ is N(R⁶)₂; R⁴ is H, C₁-C₆ alkyl optionally substituted with one or more halogen, or —C(O)—(C₁-C₆ alkyl) optionally substituted with one or more halogen; each R⁶ is independently H or C₁-C₆ alkyl optionally substituted with one or more halogen; X² is N(R⁹)₂; R⁷ is H, C₁-C₆ alkyl optionally substituted with one or more halogen, or —C(O)—(C₁-C₆ alkyl) optionally substituted with one or more halogen; and R⁹ is H or C₁-C₆ alkyl optionally substituted with one or more halogen.

In other embodiments, X¹ is N(R³)₂; R⁴ is H, methyl, ethyl, propyl, butyl, —C(O)— methyl, —C(O)-ethyl, —C(O)-propyl, or —C(O)-butyl, each optionally substituted with one or more halogen; one R⁶ is H and the other R⁶ is methyl, ethyl, or propyl, or butyl, each optionally substituted with one or more halogen; X¹ is N(R⁹)₂; R⁷ is H, methyl, ethyl, propyl, butyl, —C(O)-methyl, —C(O)-ethyl, —C(O)-propyl, or —C(O)-butyl, each optionally substituted with one or more halogen; and one R⁹ is H and the other R⁹ is methyl, ethyl, or propyl, or butyl, each optionally substituted with one or more halogen.

In other embodiments, the thiol-based compounds can have a structure of formula (III):

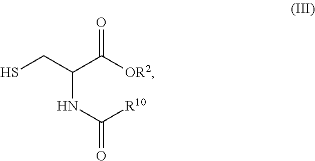

or an adduct, a pharmaceutically acceptable salt, a tautomer, or a solvate thereof; wherein
R² is H or alkyl optionally substituted with one or more halogen; and
R¹⁰ is an alkyl optionally substituted with one or more halogen.

In some embodiments, the adduct of the compounds of formula III can be a biologically active adduct and include at least one of an albumin adduct, a glucose adduct, an L-cysteine adduct, an L-glutathione adduct, an D-cysteine adduct, or an S-nitroso adduct.

In some embodiments, R² is H or C₁-C₆ alkyl optionally substituted with one or more halogen.

In other embodiments, R² is H, methyl, ethyl, or propyl, or butyl, each optionally substituted with one or more halogen.

In some embodiments, R¹⁰ is C₁-C₆ alkyl optionally substituted with one or more halogen.

In other embodiments, R¹⁰ is methyl, ethyl, or propyl, or butyl, each optionally substituted with one or more halogen.

In other embodiments, the thiol-based compounds can have a structure of formula (IV):

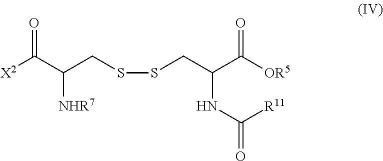

or an adduct, a pharmaceutically acceptable salt, a tautomer, or a solvate thereof; wherein
X² is OR⁸ or N(R⁹)₂;
R⁵ and R⁸ are each independently H or alkyl optionally substituted with one or more halogen;
R⁷ is H, alkyl optionally substituted with one or more halogen, or —C(O)-alkyl optionally substituted with one or more halogen;
each R⁹ is H or alkyl optionally substituted with one or more halogen; and
R¹¹ is an alkyl optionally substituted with one or more halogen.

In some embodiments, the adduct of the compounds of formula IV can be a biologically active adduct and include at least one of an albumin adduct, a glucose adduct, an L-cysteine adduct, an L-glutathione adduct, an D-cysteine adduct, or an S-nitroso adduct.

In some embodiments, R⁵ and R⁸ are each H or C₁-C₆ alkyl optionally substituted with one or more halogen.

In other embodiments, R⁵ and R⁸ are each H, methyl, ethyl, or propyl, or butyl, each optionally substituted with one or more halogen.

In some embodiments, R⁷ is H, C₁-C₆ alkyl optionally substituted with one or more halogen, or —C(O)—(C₁-C₆ alkyl) optionally substituted with one or more halogen.

In other embodiments, R⁷ is H, methyl, ethyl, propyl, butyl, —C(O)-methyl, —C(O)-ethyl, —C(O)-propyl, or —C(O)-butyl, each optionally substituted with one or more halogen.

In some embodiments, each R⁹ is independently H or C₁-C₆ alkyl optionally substituted with one or more halogen.

In other embodiments, one R⁹ is H and the other R⁹ is methyl, ethyl, or propyl, or butyl, each optionally substituted with one or more halogen.

In some embodiments, R¹¹ is C₁-C₆ alkyl optionally substituted with one or more halogen.

In other embodiments, R^{1′} is methyl, ethyl, or propyl, or butyl, each optionally substituted with one or more halogen.

In still other embodiments, the thiol-based compounds can have a structure of formula (V):

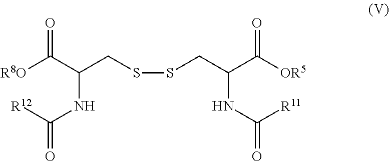

or an adduct, a pharmaceutically acceptable salt, a tautomer, or a solvate thereof; wherein
R⁵ and R⁸ are each independently H or alkyl optionally substituted with one or more halogen; and
R¹¹ and R¹² are each independently an alkyl optionally substituted with one or more halogen.

In some embodiments, the adduct of the compounds of formula V can be a biologically active adduct and include at least one of an albumin adduct, a glucose adduct, an L-cysteine adduct, an L-glutathione adduct, an D-cysteine adduct, or an S-nitroso adduct.

In some embodiments, R⁵ and R⁸ are each H or C₁-C₆ alkyl optionally substituted with one or more halogen.

In other embodiments, R⁵ and R⁸ are each H, methyl, ethyl, or propyl, or butyl, each optionally substituted with one or more halogen.

In some embodiments, R^{1′} and R¹² are each independently C₁-C₆ alkyl optionally substituted with one or more halogen.

In other embodiments, R^{1′} and R¹² are each independently methyl, ethyl, or propyl, or butyl, each optionally substituted with one or more halogen.

In other embodiments, the thiol-based compounds can have a structure of formula (VI):

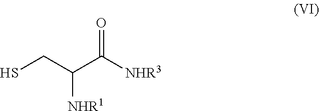

or an adduct, a pharmaceutically acceptable salt, a tautomer, or a solvate thereof; wherein:
R¹ is H, alkyl optionally substituted with one or more halogen, or —C(O)-alkyl optionally substituted with one or more halogen; and
R³ is H or alkyl optionally substituted with one or more halogen.

In some embodiments, the adduct of the compounds of formula VI can be a biologically active adduct and include at least one of an albumin adduct, a glucose adduct, an L-cysteine adduct, an L-glutathione adduct, an D-cysteine adduct, or an S-nitroso adduct.

In some embodiments, R¹ is H, C₁-C₆ alkyl optionally substituted with one or more halogen, or —C(O)—(C₁-C₆ alkyl) optionally substituted with one or more halogen.

In other embodiments, R¹ is H, methyl, ethyl, propyl, butyl, —C(O)-methyl, —C(O)-ethyl, —C(O)-propyl, or —C(O)-butyl, each optionally substituted with one or more halogen.

In some embodiments, R³ is H or C₁-C₆ alkyl optionally substituted with one or more halogen.

In other embodiments, R³ is H, methyl, ethyl, or propyl, or butyl, each optionally substituted with one or more halogen.

In other embodiments, the thiol-based compounds can have a structure of formula (VII):

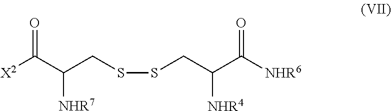

or an adduct, a pharmaceutically acceptable salt, a tautomer, or a solvate thereof; wherein
X² is OR⁸ or N(R⁹)₂;
R⁴ is H, alkyl optionally substituted with one or more halogen, or —C(O)-alkyl optionally substituted with one or more halogen;
R⁶ is H or alkyl optionally substituted with one or more halogen;
R⁷ is H, alkyl optionally substituted with one or more halogen, or —C(O)-alkyl optionally substituted with one or more halogen;
R⁸ is H or alkyl optionally substituted with one or more halogen; and
each R⁹ is H or alkyl optionally substituted with one or more halogen.

In some embodiments, the adduct of the compounds of formula (VII) can be a biologically active adduct and include at least one of an albumin adduct, a glucose adduct, an L-cysteine adduct, an L-glutathione adduct, an D-cysteine adduct, or an S-nitroso adduct.

In some embodiments, R⁴ is H, C₁-C₆ alkyl optionally substituted with one or more halogen, or —C(O)—(C₁-C₆ alkyl) optionally substituted with one or more halogen.

In other embodiments, R⁴ is H, methyl, ethyl, propyl, butyl, —C(O)-methyl, —C(O)-ethyl, —C(O)-propyl, or —C(O)-butyl, each optionally substituted with one or more halogen.

In some embodiments, R⁶ is independently H or C₁-C₆ alkyl optionally substituted with one or more halogen.

In other embodiments, R⁶ is methyl, ethyl, or propyl, or butyl, each optionally substituted with one or more halogen.

In some embodiments, R⁷ is H, C₁-C₆ alkyl optionally substituted with one or more halogen, or —C(O)—(C₁-C₆ alkyl) optionally substituted with one or more halogen.

In other embodiments, R⁷ is H, methyl, ethyl, propyl, butyl, —C(O)-methyl, —C(O)-ethyl, —C(O)-propyl, or —C(O)-butyl, each optionally substituted with one or more halogen.

In some embodiments, R⁸ is H or C₁-C₆ alkyl optionally substituted with one or more halogen.

In other embodiments, R⁸ is H, methyl, ethyl, or propyl, or butyl, each optionally substituted with one or more halogen.

In some embodiments, each R⁹ is independently H or C₁-C₆ alkyl optionally substituted with one or more halogen.

In other embodiments, one R⁹ is H and the other R⁹ is methyl, ethyl, or propyl, or butyl, each optionally substituted with one or more halogen.

In other embodiments, the thiol-based compounds can have a structure of formula (VIII):

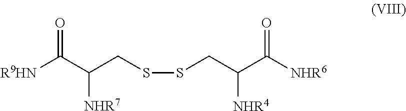

or an adduct, a pharmaceutically acceptable salt, a tautomer, or a solvate thereof; wherein
R⁴ is H, alkyl optionally substituted with one or more halogen, or —C(O)-alkyl optionally substituted with one or more halogen;
R⁶ is H or alkyl optionally substituted with one or more halogen;
R⁷ is H, alkyl optionally substituted with one or more halogen, or —C(O)-alkyl optionally substituted with one or more halogen; and
R⁹ is H or alkyl optionally substituted with one or more halogen.

In some embodiments, the adduct of the compounds of formula VIII can be a biologically active adduct and include at least one of an albumin adduct, a glucose adduct, an L-cysteine adduct, an L-glutathione adduct, an D-cysteine adduct, or an S-nitroso adduct.

In some embodiments, R⁴ is H, C₁-C₆ alkyl optionally substituted with one or more halogen, or —C(O)—(C₁-C₆ alkyl) optionally substituted with one or more halogen.

In other embodiments, R⁴ is H, methyl, ethyl, propyl, butyl, —C(O)-methyl, —C(O)-ethyl, —C(O)-propyl, or —C(O)-butyl, each optionally substituted with one or more halogen.

In some embodiments, R⁶ is independently H or C₁-C₆ alkyl optionally substituted with one or more halogen.

In other embodiments, R⁶ is methyl, ethyl, or propyl, or butyl, each optionally substituted with one or more halogen.

In some embodiments, R⁷ is H, C₁-C₆ alkyl optionally substituted with one or more halogen, or —C(O)—(C₁-C₆ alkyl) optionally substituted with one or more halogen.

In other embodiments, R⁷ is H, methyl, ethyl, propyl, butyl, —C(O)-methyl, —C(O)-ethyl, —C(O)-propyl, or —C(O)-butyl, each optionally substituted with one or more halogen.

In some embodiments, R⁹ is C₁-C₆ alkyl optionally substituted with one or more halogen.

In other embodiments, R⁹ is methyl, ethyl, or propyl, or butyl, each optionally substituted with one or more halogen.

Additional embodiments described herein relate to a pharmaceutical composition including a compound of formulas I, II, III, IV, V, VI, VII, VIII, or an adduct, a pharmaceutically acceptable salt, a tautomer, or a solvate thereof described herein and a pharmaceutically acceptable carrier or excipient. In certain embodiments, the pharmaceutical composition can be formulated for at least one of intravenous, oral, intranasal, or inhalation administration.

Other embodiments described herein relate to methods of attenuating and/or treating opioid induced ventilatory and/or respiratory depression in a subject in need thereof by administering to the subject a therapeutically effective amount of a composition comprising a compound having the structure of formulas I, II, III, IV, V, VI, VII, VIII, or an adduct, a pharmaceutically acceptable salt, a tautomer, or a solvate thereof described herein.

Other embodiments described herein relate to methods of attenuating, reducing, and/or treating opioid induced constipation and/or anuria in a subject in need thereof by administering to the subject a therapeutically effective amount of a composition comprising a compound having the structure of formulas I, II, III, IV, V, VI, VII, VIII, or an adduct, a pharmaceutically acceptable salt, a tautomer, or a solvate thereof described herein.

Still other embodiments described herein relate to methods of treating brain injury in a subject in need thereof, such as an opioid induced brain injury, by administering to the subject a therapeutically effective amount of a composition comprising a compound having the structure of formulas I, II, III, IV, V, VI, VII, VIII, or an adduct, a pharmaceutically acceptable salt, a tautomer, or a solvate thereof described herein.

In some embodiments, the method of treating brain injury in a subject in need thereof further includes the step of detecting a level of nitrotyrosine (NT) in a biological sample obtained from the subject, wherein an increase of the level of NT compared to a control is indicative of the subject having a brain injury. In some embodiments, the biological sample is selected from the group consisting of cerebral spinal fluid (CSF), saliva, and blood.

In some embodiments, the opioid can include at least one of alfentanil, buprenorphine, butorphanol, carfentanil, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanil, sufentanil, tapentadol, and tramadol, and pharmaceutically acceptable salts thereof. In other embodiments, the opioid is carfentanil, fentanyl, remifentanil, or sufentanil.

In some embodiments, the opioid administration elicits disturbances in ventilatory parameters (e.g., decreases in frequency of breathing, tidal volume, and minute ventilation), Arterial Blood Gas (ABG) chemistry (e.g., decreases in pH, pO₂, sO₂ with increases in pCO₂) and Alveolar-arterial (A-a) gradient while causing sedation and analgesia.

In other embodiments, administration of the therapeutically effective amount of the composition is effective to elicit sustained reversal of opioid elicited disturbances in ventilatory parameters (e.g., decreases in frequency of breathing, tidal volume, and minute ventilation), Arterial Blood Gas (ABG) chemistry (e.g., decreases in pH, pO₂, sO₂ with increases in pCO₂) and Alveolar-arterial (A-a) gradient while maintaining opioid sedation and analgesia.

In some embodiments, the opioid administration elicits hypoxic brain injury, front brain region impairments (e.g., decreased memory, attention, spatial planning, and executive brain function), and/or increases in nitrotyrosine (NT) while causing sedation and analgesia.

In other embodiments, administration of the therapeutically effective amount of the composition is effective to elicit sustained reversal of opioid elicited hypoxic brain injury, front brain region impairments (e.g., decreased memory, attention, spatial planning, and executive brain function), and/or increases in nitrotyrosine (NT) while causing sedation and analgesia.

In some embodiments, the composition is administered to the subject systemically by, for example, topical (e.g., inhalation), enteral (e.g., oral), and/or parenteral (e.g., intravenous injection) administration. For example, the opioid can be administered systemically by continuous intravenous infusion. In some embodiments, the composition can be administered by oral or intranasal inhalation.

In some embodiments, the composition is administered concurrently with opioid administration and/or up to about 10 minutes, up to about 20 minutes, up to about 30 minutes, up to about 40 minutes, up to about 50 minutes, up to about 60 minutes, up to about 70 minutes, up to about 80 minutes, up to about 90 minutes, up to about 100 minutes, up to about 110 minutes, or up to about 120 minutes before or after initiation of opioid administration. The opioid can be, for example, fentanyl.

In other embodiments, the composition can be administered to the subject at an amount effective to prevent the need for mechanical ventilation in subjects with acutely impaired ventilatory and/or respiratory drive because of an acute requirement for narcotic analgesia.

In still other embodiments, the composition can be administered to a subject in combination with at least one additional therapeutic agent that changes normal breathing in a subject. The additional agent can be selected from the group consisting of an opioid, doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists, ampakines, and combinations thereof.

In another embodiment, the compound and the additional therapeutic agent are separately administered to the subject. In yet another embodiment, the compound and the additional therapeutic agent are co-administered to the subject.

Still other embodiments described herein relate to a composition that includes an opioid capable of inducing ventilatory and/or respiratory depression in a subject and an amount of thiol-based compound having the structure of formulas I, II, III, IV, V, VI, VII, VIII, or an adduct, a pharmaceutically acceptable salt, a tautomer, or a solvate thereof described herein effective to attenuate the opioid induced ventilatory and/or respiratory depression when the composition is administered to the subject.

In some embodiments, the opioid can include at least one of alfentanil, buprenorphine, butorphanol, carfentanil, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanil, sufentanil, tapentadol, and tramadol, and pharmaceutically acceptable salts thereof. For example, the opioid can be carfentanil, fentanyl, remifentanil, or sufentanil.

### BRIEF DESCRIPTION OF THE DRAWINGS

FIGS. 1(A-C) illustrate plots showing L-NAC reverses the adverse effects of fentanyl infusion on Freq, TV and MV. A summary of the values of frequency of breathing (Panel A), tidal volume (Panel B) and minute ventilation (Panel C) before (Pre) and during the continuous intravenous infusion of fentanyl (1 μg/kg/min) and following two bolus intravenous injections of vehicle (VEH) or N-acetyl-L-cysteine (L-NAC, 500 mol/kg) given at 60 and 90 min of fentanyl infusion in two groups of unanesthetized rats. There were 6 rats in each group. The data are presented as mean±SEM.

FIGS. 2(A-C) illustrate plots showing L-NAC reverses the adverse effects of fentanyl infusion on Ti, Te and Ti/Te. A summary of the values of inspiratory time (Ti, Panel A), expiratory time (Te, Panel B) and Ti/Te (Panel C) before (Pre) and during continuous intravenous infusion of fentanyl (1 μg/kg/min) and following two bolus intravenous injections of vehicle (VEH) or N-acetyl-L-cysteine (L-NAC, 500 mol/kg) given at 60 and 90 min of fentanyl infusion in two groups of unanesthetized rats. There were 6 rats in each group. The data are presented as mean±SEM.

FIGS. 3(A-B) illustrate plots showing L-NAC reverses the adverse effects of fentanyl infusion on EEP but not EIP. A summary of the values of end inspiratory pause (Panel A) and end expiratory pause (Panel B) before (Pre) and during continuous intravenous infusion of fentanyl (1 μg/kg/min) and following two bolus intravenous injections of vehicle (VEH) or N-acetyl-L-cysteine (L-NAC, 500 mol/kg) given at 60 and 90 min of fentanyl infusion in two groups of unanesthetized rats. There were 6 rats in each group. The data are presented as mean±SEM.

FIG. 4(A-C) illustrate plots showing L-NAC reverses the adverse effects of fentanyl infusion on PIF and PEF. A summary of the values of peak inspiratory flow (PIF, Panel A), peak expiratory flow (PEF, Panel B) and PIF/PEF (Panel C) before (Pre) and during continuous intravenous infusion of fentanyl (1 μg/kg/min) and following two bolus intravenous injections of vehicle (VEH) or N-acetyl-L-cysteine (L-NAC, 500 μmol/kg) given at 60 and 90 min of fentanyl infusion in two groups of unanesthetized rats. There were 6 rats in each group. The data are presented as mean±SEM.

FIGS. 5(A-B) illustrate plots showing L-NAC reverses the adverse effects of fentanyl infusion on inspiratory and expiratory drives. A summary of the values of inspiratory drive (TV/Ti, Panel A) and expiratory drive (TV/Te, Panel B) before (Pre) and during continuous intravenous infusion of fentanyl (1 μg/kg/min) and following two bolus intravenous injections of vehicle (VEH) or N-acetyl-L-cysteine (L-NAC, 500 mol/kg) given at 60 and 90 min of fentanyl infusion in two groups of unanesthetized rats. There were 6 rats in each group. The data are presented as mean±SEM.

FIGS. 6(A-C) illustrate plots showing L-NAC reverses adverse effects of fentanyl infusion on expiratory delay and expiratory ratio. A summary of the values of relaxation time flow (RT, Panel A), expiratory delay (Te-RT, Panel B) and expiratory ratio [(Te/RT)−1, Panel C] before (Pre) and during continuous intravenous infusion of fentanyl (1 μg/kg/min) and after two bolus intravenous injections of vehicle (VEH) or N-acetyl-L-cysteine (L-NAC, 500 μmol/kg) given at 60 and 90 min of fentanyl infusion in two groups of unanesthetized rats. There were 6 rats in each group. The data are presented as mean±SEM.

FIGS. 7(A-C) illustrate graphs showing the first injection of L-NAC reverses many adverse effects of fentanyl infusion on ventilatory parameters. A summary of total (cumulative) changes in Freq, TV, MV, Ti, Te, Ti/Te, EIP, EEP (Panel A), PIF, PEF, PIF/PEF, EF50, InspD, ExpD, NEBI (Panel B) and expiratory pause and Te-RT (Panel C) during the 30 min period after the first injection of vehicle or N-acetyl-L-cysteine (L-NAC, 500 mol/kg, IV) in rats receiving a continuous infusion of fentanyl (1 μg/kg/min, IV). There were 6 rats in each group. data are presented as mean±SEM. *P<0.05, significant change from pre-fentanyl values. †P<0.05, L-NAC versus pre.

FIGS. 8(A-C) illustrate graphs showing L-NAC reverses the adverse effects of fentanyl infusion on ABG chemistry and A-a gradient. A summary of the arithmetic changes in pH (Panel A), pCO₂ (Panel B), PO₂ (Panel C), sO₂ (Panel D), and Alveolar-arterial (A-a) gradient (Panel E) elicited by continuous infusion of fentanyl (1 μg/kg/min, IV) and bolus injections of vehicle or N-acetyl-L-cysteine (L-NAC, 500 μmol/kg, IV) in unanesthetized rats. There were 6 rats in each group. The data are presented as mean±SEM. *P<0.05, significant change from pre-fentanyl values. †P<0.05, L-NAC versus pre.

FIGS. 9(A-E) illustrate plots showing L-NAC reverses the adverse effects of fentanyl infusion on ABG chemistry and A-a gradient. A summary of the arithmetic changes in pH (Panel A), pCO₂ (Panel B), PO₂ (Panel C), sO₂ (Panel D), and Alveolar-arterial (A-a) gradient (Panel E) elicited by continuous infusion of fentanyl (1 μg/kg/min, IV) and bolus injections of vehicle or N-acetyl-L-cysteine (L-NAC, 500 μmol/kg, IV) in unanesthetized rats. There were 6 rats in each group. The data are presented as mean±SEM. *P<0.05, significant change from pre-fentanyl values. †P<0.05, L-NAC versus pre.

FIG. 10 illustrates a plot showing the relationships between peak inspiratory flow (PIF), peak expiratory flow (PEF), relaxation time (RT) and expiratory time (Te).

FIGS. 11(A-B) illustrate plots showing L-NAC reverses the adverse effects of fentanyl infusion on EF₅₀ and Rpef. A summary of values of expiratory flow at 50% tidal volume (EF₅₀, Panel A) and non-eupneic breathing index (NEBI, Panel B) before (Pre) and during the continuous intravenous infusion of fentanyl (1 μg/kg/min) and following two bolus intravenous injections of vehicle (VEH) or N-acetyl-L-cysteine (L-NAC, 500 mol/kg) given at 60 and 90 min of fentanyl infusion in two groups of unanesthetized rats. There were 6 rats in each group. The data are presented as mean±SEM.

FIG. 12 illustrates plots showing changes in frequency of breathing (top panel), tidal volume (middle panel) and minute ventilation (bottom panel) in freely moving rats upon (a) injection of morphine (10 mg/kg, IV) and two subsequent injections of vehicle (saline) or N-acetyl-L-cysteine ethyl ester (L-NACme; 500 mol/kg, IV). The data are shown as mean±SEM. There were 9 rats in each group.

FIGS. 13(A-D) illustrate graphs showing Panel A: Body weights. Panel B. Urine output. Panel C. Number of fecal pellets. Panel D. Dry weight of the collected fecal pellets. Data are shown as mean±SEM, there were 9 rats in each group. *P<0.05, significant difference from zero. †P<0.05, significant difference between fentanyl group and other groups.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the present invention.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or” unless the context clearly indicates otherwise.

The term “about” or “approximately” as used herein refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, 10%, ±9%, ±8%, ±7%, ±6%, ±5%, ±4%, ±3%, ±2%, or ±1% about a reference quantity, level, value, number, frequency, percentage, dimension, size, amount, weight or length.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

The term “pharmaceutical composition” refers to a formulation containing the disclosed compounds in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In some embodiments, the compound or active ingredient is mixed under sterile conditions with a pharmaceutically acceptable carrier, and with any preservatives, buffers, or propellants that are required.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials, which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compounds of the application are capable of further forming salts. All of these forms are also contemplated herein.

“Pharmaceutically acceptable salt” of a compound means a salt that is pharmaceutically acceptable and that possesses the desired pharmacological activity of the parent compound. For example, the salt can be an acid addition salt. One embodiment of an acid addition salt is a hydrochloride salt. The pharmaceutically acceptable salts can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile being preferred. Lists of salts are found in Remington's Pharmaceutical Sciences, 18th ed. (Mack Publishing Company, 1990).

The compounds described herein can also be prepared as esters, for example pharmaceutically acceptable esters. For example, a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds described herein can also be prepared as prodrugs, for example pharmaceutically acceptable prodrugs. The terms “pro-drug” and “prodrug” are used interchangeably herein and refer to any compound, which releases an active parent drug in vivo. Since prodrugs are known to enhance numerous desirable qualities of pharmaceuticals (e.g., solubility, bioavailability, manufacturing, etc.) the compounds can be delivered in prodrug form. Thus, the compounds described herein are intended to cover prodrugs of the presently claimed compounds, methods of delivering the same and compositions containing the same. “Prodrugs” are intended to include any covalently bonded carriers that release an active parent drug in vivo when such prodrug is administered to a subject. Prodrugs are prepared by modifying functional groups present in the compound in such a way that the modifications are cleaved, either in routine manipulation or in vivo, to the parent compound. Prodrugs include compounds wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively. Prodrugs can also include a precursor (forerunner) of a compound described herein that undergoes chemical conversion by metabolic processes before becoming an active or more active pharmacological agent or active compound described herein.

Additionally, the salts of the compounds described herein, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

The term “solvates” means solvent addition forms that contain either stoichiometric or nonstoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds, salts and prodrugs described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

A “patient,” “subject,” or “host” to be treated by the compounds or methods described herein may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compounds. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament”, “active ingredient”, and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

With respect to any chemical compounds, the present application is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

When an atom or a chemical moiety is followed by a subscripted numeric range (e.g., C₁₋₆), it is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

The term “alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅alkyl includes C₅ alkyls, C₄ alkyls, C₃ alkyls, C₂ alkyls and C₁ alkyl (i.e., methyl). A C₁-C₆alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

All percentages and ratios used herein, unless otherwise indicated, are by weight.

Embodiments described herein relate to thiol-based compounds and pharmaceutical compositions thereof, and to their use in methods of attenuating opioid induced ventilatory and/or respiratory depression, attenuating opioid induced constipation and/or anuria, and/or treating brain injury in a subject in need thereof.

In some embodiments, the thiol-based compounds can have a structure of formulas:

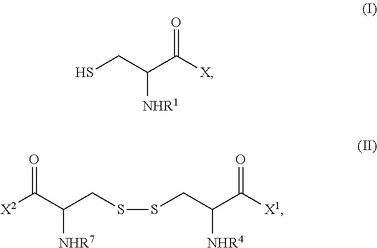

or an adduct, a pharmaceutically acceptable salt, a tautomer, or a solvate thereof; wherein:
X is OR² or N(R³)₂;
X¹ is OR⁵ or N(R⁶)₂;
X² is OR⁸ or N(R⁹)₂;
R¹, R⁴, and R⁷ are each independently H, alkyl optionally substituted with one or more halogen, or —C(O)-alkyl optionally substituted with one or more halogen;
R², R⁵, and R⁸ are each independently H or alkyl optionally substituted with one or more halogen;
each R³, R⁶, and R⁹ are independently H or alkyl optionally substituted with one or more halogen;
X is not OR² if R¹ is H; and
X² is not OR⁸ if X¹ is OR⁵ and R⁴ and R⁷ are H.

In some embodiments, the adduct of the compounds of formulas I or II can be a biologically active adduct and include at least one of an albumin adduct, a glucose adduct, an L-cysteine adduct, an L-glutathione adduct, an D-cysteine adduct, or an S-nitroso adduct.

In other embodiments, the compound is not cysteine, cystine, a cysteine alkylester, or cystine dialkylester.

In some embodiments, R¹, R⁴, and R⁷ are each independently H, C₁-C₆ alkyl optionally substituted with one or more halogen, or —C(O)—(C₁-C₆ alkyl) optionally substituted with one or more halogen.

In other embodiments, R¹, R⁴, and R⁷ are each independently H, methyl, ethyl, propyl, butyl, —C(O)-methyl, —C(O)-ethyl, —C(O)-propyl, or —C(O)-butyl, each optionally substituted with one or more halogen.

In some embodiments, R², R⁵, and R⁸ are each independently H or C₁-C₆ alkyl optionally substituted with one or more halogen.

In other embodiments, R², R⁵, and R⁸ are each independently H, methyl, ethyl, or propyl, or butyl, each optionally substituted with one or more halogen.

In some embodiments, each R³, R⁶, and R⁹ is independently H or C₁-C₆ alkyl optionally substituted with one or more halogen.

In other embodiments, one of each R³, R⁶, and R⁹ is H and the other of R³, R⁶, and R⁹ is methyl, ethyl, or propyl, or butyl, each optionally substituted with one or more halogen.

In some embodiments, X is OR²; R¹ is H, C₁-C₆ alkyl optionally substituted with one or more halogen, or —C(O)—(C₁-C₆ alkyl) optionally substituted with one or more halogen; and R² is H or C₁-C₆ alkyl optionally substituted with one or more halogen.

In other embodiments, X is OR²; R¹ is H, methyl, ethyl, propyl, butyl, —C(O)— methyl, —C(O)-ethyl, —C(O)-propyl, or —C(O)-butyl, each optionally substituted with one or more halogen; and R² is H, methyl, ethyl, or propyl, or butyl, each optionally substituted with one or more halogen.

In some embodiments, X is N(R³)₂; R¹ is H, C₁-C₆ alkyl optionally substituted with one or more halogen, or —C(O)—(C₁-C₆ alkyl) optionally substituted with one or more halogen; and each R³ is independently H or C₁-C₆ alkyl optionally substituted with one or more halogen.

In other embodiments, X is N(R³)₂; R¹ is H, methyl, ethyl, propyl, butyl, —C(O)— methyl, —C(O)-ethyl, —C(O)-propyl, or —C(O)-butyl, each optionally substituted with one or more halogen; and one R³ is H and the other R³ is methyl, ethyl, or propyl, or butyl, each optionally substituted with one or more halogen.

In still other embodiments, X¹ is OR⁵; R⁴ is H, C₁-C₆ alkyl optionally substituted with one or more halogen, or —C(O)—(C₁-C₆ alkyl) optionally substituted with one or more halogen; R⁵ is H or C₁-C₆ alkyl optionally substituted with one or more halogen; X² is OR⁸; R⁷ is H, C₁-C₆ alkyl optionally substituted with one or more halogen, or —C(O)—(C₁-C₆ alkyl) optionally substituted with one or more halogen; and R⁸ is H or C₁-C₆ alkyl optionally substituted with one or more halogen.

In other embodiments, X¹ is OR⁵; R⁴ is H, methyl, ethyl, propyl, butyl, —C(O)— methyl, —C(O)-ethyl, —C(O)-propyl, or —C(O)-butyl, each optionally substituted with one or more halogen; R⁵ is H, methyl, ethyl, or propyl, or butyl, each optionally substituted with one or more halogen; X² is OR⁸; R⁷ is H, methyl, ethyl, propyl, butyl, —C(O)-methyl, —C(O)-ethyl, —C(O)-propyl, or —C(O)-butyl, each optionally substituted with one or more halogen; and R⁸ is H, methyl, ethyl, or propyl, or butyl, each optionally substituted with one or more halogen.

In some embodiments, X¹ is N(R⁶)₂; R⁴ is H, C₁-C₆ alkyl optionally substituted with one or more halogen, or —C(O)—(C₁-C₆ alkyl) optionally substituted with one or more halogen; each R⁶ is independently H or C₁-C₆ alkyl optionally substituted with one or more halogen; X² is N(R⁹)₂; R⁷ is H, C₁-C₆ alkyl optionally substituted with one or more halogen, or —C(O)—(C₁-C₆ alkyl) optionally substituted with one or more halogen; and R⁹ is H or C₁-C₆ alkyl optionally substituted with one or more halogen.

In other embodiments, X¹ is N(R³)₂; R⁴ is H, methyl, ethyl, propyl, butyl, —C(O)— methyl, —C(O)-ethyl, —C(O)-propyl, or —C(O)-butyl, each optionally substituted with one or more halogen; one R⁶ is H and the other R⁶ is methyl, ethyl, or propyl, or butyl, each optionally substituted with one or more halogen; X¹ is N(R⁹)₂; R⁷ is H, methyl, ethyl, propyl, butyl, —C(O)-methyl, —C(O)-ethyl, —C(O)-propyl, or —C(O)-butyl, each optionally substituted with one or more halogen; and one R⁹ is H and the other R⁹ is methyl, ethyl, or propyl, or butyl, each optionally substituted with one or more halogen.

In other embodiments, the thiol-based compounds can have a structure of formula (III):

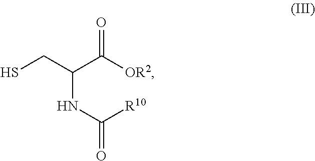

or an adduct, a pharmaceutically acceptable salt, a tautomer, or a solvate thereof; wherein
R² is H or alkyl optionally substituted with one or more halogen; and
R¹⁰ is an alkyl optionally substituted with one or more halogen.

In some embodiments, the adduct of the compounds of formula III can be a biologically active adduct and include at least one of an albumin adduct, a glucose adduct, an L-cysteine adduct, an L-glutathione adduct, an D-cysteine adduct, or an S-nitroso adduct.

In some embodiments, R² is H or C₁-C₆ alkyl optionally substituted with one or more halogen.

In other embodiments, R² is H, methyl, ethyl, or propyl, or butyl, each optionally substituted with one or more halogen.

In some embodiments, R¹⁰ is C₁-C₆ alkyl optionally substituted with one or more halogen.

In other embodiments, R¹⁰ is methyl, ethyl, or propyl, or butyl, each optionally substituted with one or more halogen.

In other embodiments, the compound of formula (III) has the structure selected from:

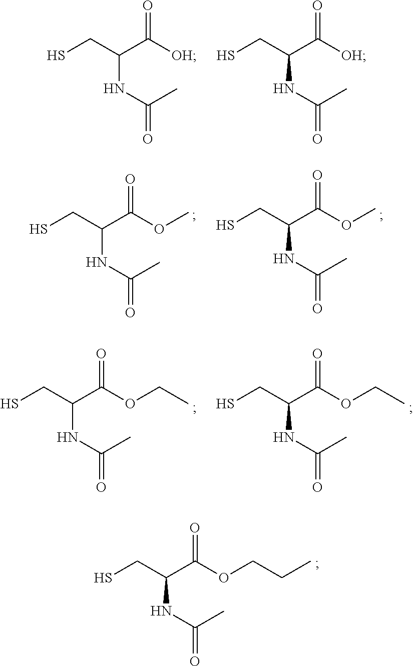

or an adduct, a pharmaceutically acceptable salt, a tautomer, or a solvate thereof.

In other embodiments, the thiol-based compounds can have a structure of formula (IV):

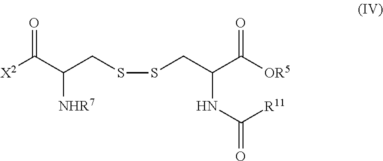

or an adduct, a pharmaceutically acceptable salt, a tautomer, or a solvate thereof; wherein
X² is OR⁸ or N(R⁹)₂;
R⁵ and R⁸ are each independently H or alkyl optionally substituted with one or more halogen;
R⁷ is H, alkyl optionally substituted with one or more halogen, or —C(O)-alkyl optionally substituted with one or more halogen;
each R⁹ is H or alkyl optionally substituted with one or more halogen; and
R¹¹ is an alkyl optionally substituted with one or more halogen.

In some embodiments, the adduct of the compounds of formula IV can be a biologically active adduct and include at least one of an albumin adduct, a glucose adduct, an L-cysteine adduct, an L-glutathione adduct, an D-cysteine adduct, or an S-nitroso adduct.

In some embodiments, R⁵ and R⁸ are each H or C₁-C₆ alkyl optionally substituted with one or more halogen.

In other embodiments, R⁵ and R⁸ are each H, methyl, ethyl, or propyl, or butyl, each optionally substituted with one or more halogen.

In some embodiments, R⁷ is H, C₁-C₆ alkyl optionally substituted with one or more halogen, or —C(O)—(C₁-C₆ alkyl) optionally substituted with one or more halogen.

In other embodiments, R⁷ is H, methyl, ethyl, propyl, butyl, —C(O)-methyl, —C(O)-ethyl, —C(O)-propyl, or —C(O)-butyl, each optionally substituted with one or more halogen.

In some embodiments, each R⁹ is independently H or C₁-C₆ alkyl optionally substituted with one or more halogen.

In other embodiments, one R⁹ is H and the other R⁹ is methyl, ethyl, or propyl, or butyl, each optionally substituted with one or more halogen.

In some embodiments, R¹¹ is C₁-C₆ alkyl optionally substituted with one or more halogen.

In other embodiments, R¹¹ is methyl, ethyl, or propyl, or butyl, each optionally substituted with one or more halogen.

In still other embodiments, the thiol-based compounds can have a structure of formula (V):

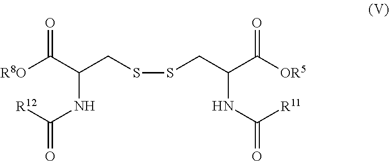

or an adduct, a pharmaceutically acceptable salt, a tautomer, or a solvate thereof; wherein
R⁵ and R⁸ are each independently H or alkyl optionally substituted with one or more halogen; and
R¹¹ and R¹² are each independently an alkyl optionally substituted with one or more halogen.

In some embodiments, the adduct of the compounds of formula V can be a biologically active adduct and include at least one of an albumin adduct, a glucose adduct, an L-cysteine adduct, an L-glutathione adduct, an D-cysteine adduct, or an S-nitroso adduct.

In some embodiments, R⁵ and R⁸ are each H or C₁-C₆ alkyl optionally substituted with one or more halogen.

In other embodiments, R⁵ and R⁸ are each H, methyl, ethyl, or propyl, or butyl, each optionally substituted with one or more halogen.

In some embodiments, R¹¹ and R¹² are each independently C₁-C₆ alkyl optionally substituted with one or more halogen.

In other embodiments, R¹¹ and R¹² are each independently methyl, ethyl, or propyl, or butyl, each optionally substituted with one or more halogen.

In other embodiments, the compound of formula (V) has the structure selected from:

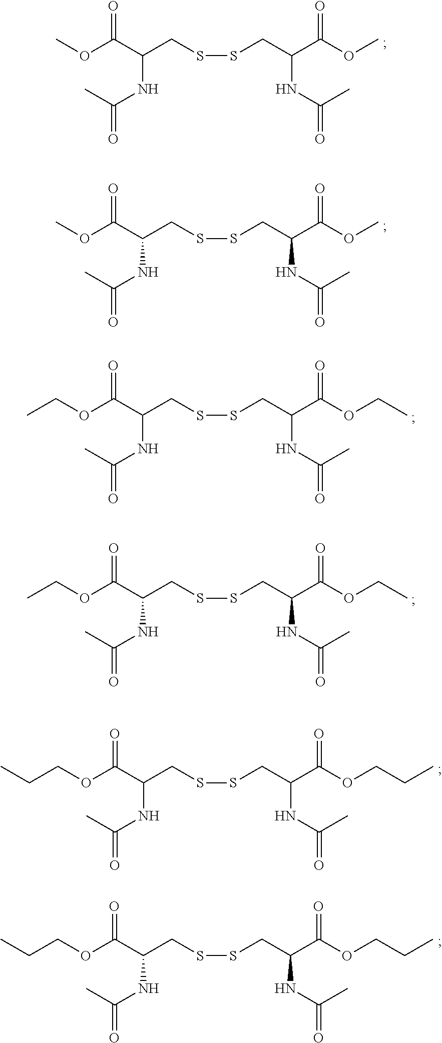

or an adduct, a pharmaceutically acceptable salt, a tautomer, or a solvate thereof.

In other embodiments, the thiol-based compounds can have a structure of formula (VI):

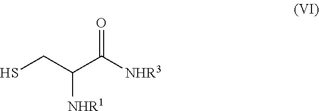

or an adduct, a pharmaceutically acceptable salt, a tautomer, or a solvate thereof; wherein:
R¹ is H, alkyl optionally substituted with one or more halogen, or —C(O)-alkyl optionally substituted with one or more halogen; and
R³ is H or alkyl optionally substituted with one or more halogen.

In some embodiments, the adduct of the compounds of formula Vi can be a biologically active adduct and include at least one of an albumin adduct, a glucose adduct, an L-cysteine adduct, an L-glutathione adduct, an D-cysteine adduct, or an S-nitroso adduct.

In some embodiments, R¹ is H, C₁-C₆ alkyl optionally substituted with one or more halogen, or —C(O)—(C₁-C₆ alkyl) optionally substituted with one or more halogen.

In other embodiments, R¹ is H, methyl, ethyl, propyl, butyl, —C(O)-methyl, —C(O)-ethyl, —C(O)-propyl, or —C(O)-butyl, each optionally substituted with one or more halogen.

In some embodiments, R³ is H or C₁-C₆ alkyl optionally substituted with one or more halogen.

In other embodiments, R³ is H, methyl, ethyl, or propyl, or butyl, each optionally substituted with one or more halogen.

In other embodiments, the compound of formula (VI) has the structure selected from:

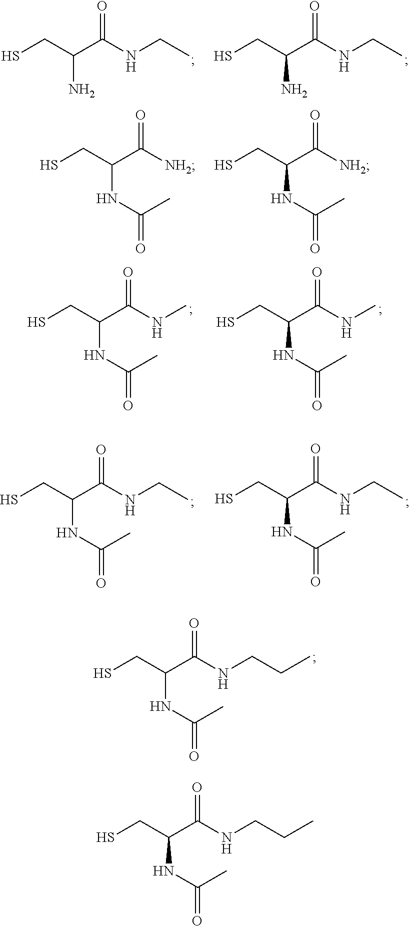

or an adduct, a pharmaceutically acceptable salt, a tautomer, or a solvate thereof.

In other embodiments, the thiol-based compounds can have a structure of formula (VII):

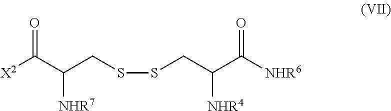

or an adduct, a pharmaceutically acceptable salt, a tautomer, or a solvate thereof; wherein
X² is OR⁸ or N(R⁹)₂;
R⁴ is H, alkyl optionally substituted with one or more halogen, or —C(O)-alkyl optionally substituted with one or more halogen;
R⁶ is H or alkyl optionally substituted with one or more halogen;
R⁷ is H, alkyl optionally substituted with one or more halogen, or —C(O)-alkyl optionally substituted with one or more halogen;
R⁸ is H or alkyl optionally substituted with one or more halogen; and
each R⁹ is H or alkyl optionally substituted with one or more halogen.

In some embodiments, the adduct of the compounds of formula (VII) can be a biologically active adduct and include at least one of an albumin adduct, a glucose adduct, an L-cysteine adduct, an L-glutathione adduct, an D-cysteine adduct, or an S-nitroso adduct.

In some embodiments, R⁴ is H, C₁-C₆ alkyl optionally substituted with one or more halogen, or —C(O)—(C₁-C₆ alkyl) optionally substituted with one or more halogen.

In other embodiments, R⁴ is H, methyl, ethyl, propyl, butyl, —C(O)-methyl, —C(O)-ethyl, —C(O)-propyl, or —C(O)-butyl, each optionally substituted with one or more halogen.

In some embodiments, R⁶ is independently H or C₁-C₆ alkyl optionally substituted with one or more halogen.

In other embodiments, R⁶ is methyl, ethyl, or propyl, or butyl, each optionally substituted with one or more halogen.

In some embodiments, R⁷ is H, C₁-C₆ alkyl optionally substituted with one or more halogen, or —C(O)—(C₁-C₆ alkyl) optionally substituted with one or more halogen.

In other embodiments, R⁷ is H, methyl, ethyl, propyl, butyl, —C(O)-methyl, —C(O)-ethyl, —C(O)-propyl, or —C(O)-butyl, each optionally substituted with one or more halogen.

In some embodiments, R⁸ is H or C₁-C₆ alkyl optionally substituted with one or more halogen.

In other embodiments, R⁸ is H, methyl, ethyl, or propyl, or butyl, each optionally substituted with one or more halogen.

In some embodiments, each R⁹ is independently H or C₁-C₆ alkyl optionally substituted with one or more halogen.

In other embodiments, R⁹ is H and the other R⁹ is methyl, ethyl, or propyl, or butyl, each optionally substituted with one or more halogen.

In other embodiments, the thiol-based compounds can have a structure of formula (VIII):

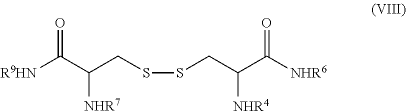

or an adduct, a pharmaceutically acceptable salt, a tautomer, or a solvate thereof; wherein
R⁴ is H, alkyl optionally substituted with one or more halogen, or —C(O)-alkyl optionally substituted with one or more halogen;
R⁶ is H or alkyl optionally substituted with one or more halogen;
R⁷ is H, alkyl optionally substituted with one or more halogen, or —C(O)-alkyl optionally substituted with one or more halogen; and
R⁹ is H or alkyl optionally substituted with one or more halogen.

In some embodiments, the adduct of the compounds of formula VIII can be a biologically active adduct and include at least one of an albumin adduct, a glucose adduct, an L-cysteine adduct, an L-glutathione adduct, an D-cysteine adduct, or an S-nitroso adduct.

In some embodiments, R⁴ is H, C₁-C₆ alkyl optionally substituted with one or more halogen, or —C(O)—(C₁-C₆ alkyl) optionally substituted with one or more halogen.

In other embodiments, R⁴ is H, methyl, ethyl, propyl, butyl, —C(O)-methyl, —C(O)-ethyl, —C(O)-propyl, or —C(O)-butyl, each optionally substituted with one or more halogen.

In some embodiments, R⁶ is independently H or C₁-C₆ alkyl optionally substituted with one or more halogen.

In other embodiments, R⁶ is methyl, ethyl, or propyl, or butyl, each optionally substituted with one or more halogen.

In some embodiments, R⁷ is H, C₁-C₆ alkyl optionally substituted with one or more halogen, or —C(O)—(C₁-C₆ alkyl) optionally substituted with one or more halogen.

In other embodiments, R⁷ is H, methyl, ethyl, propyl, butyl, —C(O)-methyl, —C(O)-ethyl, —C(O)-propyl, or —C(O)-butyl, each optionally substituted with one or more halogen.

In some embodiments, R⁹ is C₁-C₆ alkyl optionally substituted with one or more halogen.

In other embodiments, R⁹ is methyl, ethyl, or propyl, or butyl, each optionally substituted with one or more halogen.

In other embodiments, the compound of formula (VIII) has the structure selected from:

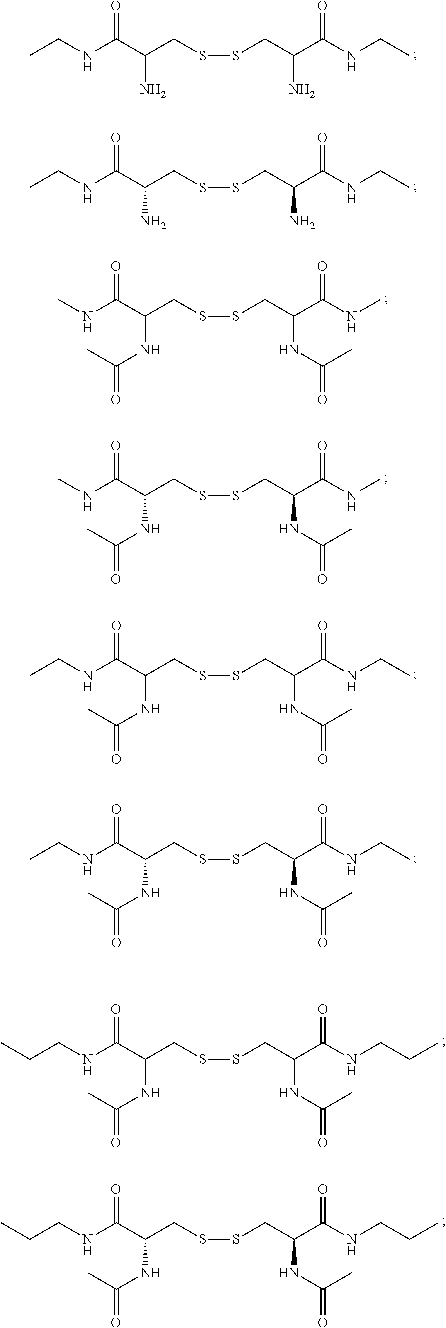

or an adduct, a pharmaceutically acceptable salt, a tautomer, or a solvate thereof.

Other embodiments described herein relate to a pharmaceutical composition including a compound of formulas I, II, III, IV, V, VI, VII, VIII, or an adduct, a pharmaceutically acceptable salt, a tautomer, or a solvate thereof described herein. In certain embodiments, the pharmaceutical composition can be formulated for at least one of intravenous, oral, intranasal, or inhalation administration as described further below.

Additional embodiments provided herein related to the therapeutic use of thiol-based compounds of formulas I, II, III, IV, V, VI, VII, VIII, or an adduct, a pharmaceutically acceptable salt, a tautomer, or a solvate thereof described herein. It was found that administration of thiol-based compounds described herein markedly attenuated the ventilatory and/or respiratory depressant effects elicited by opioids, such as fentanyl, without affecting opioid-induced analgesia. By way of example, bolus injections of a thiol-based compound, such as N-acetyl-L-cysteine (L-NAC) elicited rapid and sustained reversal of many of the adverse effects of fentanyl infusion on ventilatory parameters, ABG chemistry and A-a gradient infusion in unanesthetized male Sprague-Dawley rats without affecting sedative and antinociceptive effects of fentanyl infusion. As such, it is evident that doses of L-NAC do not sufficiently rise to levels that may directly block opioid receptors or the signaling pathways that mediate these actions of fentanyl and provide direct evidence that L-NAC interferes with opioid-receptor signaling pathways that elicit the depression of breathing and alveolar gas exchange during continuous exposure to fentanyl.

Accordingly, in some embodiments, a method of attenuating opioid induced ventilatory and/or respiratory depression in a subject in need thereof includes administering to the subject a therapeutically effective amount of a composition comprising a compound having the structure of formulas I, II, III, IV, V, VI, VII, VIII, or an adduct, a pharmaceutically acceptable salt, a tautomer, or a solvate thereof described herein.

In some embodiments, the opioid can include at least one of alfentanil, buprenorphine, butorphanol, carfentanil, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanil, sufentanil, tapentadol, and tramadol, and pharmaceutically acceptable salts thereof.

In other embodiments, the opioid is carfentanil, fentanyl, remifentanil, or sufentanil.

In some embodiments, the opioid administration elicits disturbances in ventilatory parameters (e.g., decreases in frequency of breathing, tidal volume, and minute ventilation), Arterial Blood Gas (ABG) chemistry (e.g., decreases in pH, pO₂, sO₂ with increases in pCO₂) and Alveolar-arterial (A-a) gradient while causing sedation and analgesia.

The amount or therapeutically effective amount of the composition including the thiol-based compound described herein administered to the subject can be effective to elicit sustained reversal of opioid elicited disturbances in ventilatory parameters (e.g., decreases in frequency of breathing, tidal volume, and minute ventilation), Arterial Blood Gas (ABG) chemistry (e.g., decreases in pH, pO₂, sO₂ with increases in pCO₂) and Alveolar-arterial (A-a) gradient while maintaining opioid sedation and analgesia. For example, the amount or therapeutically effective amount of the composition including the thiol-based compound described herein that is administered to the subject can be an amount effective to increase tidal volume (TV), increase respiratory frequency (Freq), increase minute ventilation (MV), decrease expiratory time (TE), and increase peak expiratory flow (PEF), increase pH, increase pO₂, increase sO₂, decrease pCO₂, and/or decrease A-a gradient in the subject. For example, an opioid upon administration to a subject can depress or decrease tidal volume (TV), respiratory frequency (Freq), minute ventilation (MV), peak expiratory flow (PEF), pH, pO₂ and/or sO₂ and/or increase expiratory time (TE), pCO₂, and/or A-a gradient at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80% or more, and the thiol-based compound described herein can be administered to the subject at an amount effective to increase tidal volume (TV), respiratory frequency (Freq), minute ventilation (MV), peak expiratory flow (PEF), pH, pO₂ and/or sO₂ and/or decrease expiratory time (TE), pCO₂, and/or A-a gradient at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80% or more.

In some embodiments, the composition including the thiol-based compounds having the structure of formulas I, II, III, IV, V, VI, VII, VIII, or an adduct, a pharmaceutically acceptable salt, a tautomer, or a solvate thereof described herein can be administered to the subject to prevent the need for mechanical ventilation in subjects with acutely impaired ventilatory and/or respiratory drive because of an acute exacerbation of an underlying lung disease or an acute requirement for narcotic analgesia. For example, the subjects can be at-risk subjects with severe, hypercapneic COPD or mixed apnea evident on polysomnography.

In other embodiments, a composition including the thiol-based compounds of formulas I, II, III, IV, V, VI, VII, VIII, or an adduct, a pharmaceutically acceptable salt, a tautomer, or a solvate thereof described herein can be administered in ambulatory delivery formulations to treat respiratory depression associated with narcotics, analgesics, sedatives, and/or opioids. The subject can be one who is taking and/or over-dosed on the narcotics, analgesics, sedatives, and/or opioids and who is experiencing or at risk of acute ventilatory and/or respiratory depression.

In some embodiments, a subject can include a subject with an increased risk of decreased ventilatory and/or respiratory drive such as a subject with a significant chronic obstructive pulmonary disease, and those with a substantially decreased respiratory reserve, hypoxia, hypercapnia, or pre-existing respiratory depression. Elderly, cachectic, or debilitated subjects may have altered pharmacokinetics or altered opioid clearance compared to younger, healthier patients resulting in greater risk for respiratory depression.

Other embodiments described herein relate to compositions and methods of attenuating, reducing, and/or treating opioid induced constipation and/or anuria in a subject in need thereof, and particularly relates to the use of thiol-based compounds in compositions and methods of attenuating, reducing, and/or treating opioid induced constipation and/or anuria in a subject in need thereof. It was found that administration of thiol-based compounds described herein markedly attenuated or reduced constipation and/or anuria effects elicited by opioids, such as fentanyl, without affecting opioid-induced analgesia. As discussed in Example 2, bolus injections of N-acetyl-L-cysteine (L-NAC) were found to overcome the constipation and anuria (loss of urine excretion) elicited by the infusion of fentanyl in rats.

Accordingly, in some embodiments, a method of attenuating, reducing, and/or treating opioid induced constipation and/or anuria in a subject in need thereof includes administering to the subject a therapeutically effective amount of a composition comprising a compound having the structure of formulas I, II, III, IV, V, VI, VII, VIII, or an adduct, a pharmaceutically acceptable salt, a tautomer, or a solvate thereof described herein.

Other embodiments described herein relate to compositions and methods of treating brain injury in a subject in need thereof, and particularly relates to the use of thiol-based compounds of formulas I, II, III, IV, V, VI, VII, VIII, or an adduct, a pharmaceutically acceptable salt, a tautomer, or a solvate thereof described herein in compositions and methods of treating brain injury in a subject in need thereof.

Accordingly, in some embodiments, a method of treating brain injury in a subject in need thereof includes administering to the subject a therapeutically effective amount of a composition comprising a compound having the structure of formulas I, II, III, IV, V, VI, VII, VIII, or an adduct, a pharmaceutically acceptable salt, a tautomer, or a solvate thereof described herein.

In some embodiments, the brain injury can include an opioid induced brain injury. In some embodiments, the opioid administration elicits hypoxic brain injury, front brain region impairments (e.g., decreased memory, attention, spatial planning, and executive brain function), and/or increases in nitrotyrosine (NT) while causing sedation and analgesia. The term “nitrotyrosine” or “NT” as used herein refers to the modification of tyrosine residues in proteins to create 3-nitro-L-tyrosine residues. Peroxynitrite reacts with the phenolic ring of tyrosine residues in proteins to create this stable adduct.

The amount or therapeutically effective amount of the composition including the thiol-based compounds described herein administered to the subject can be effective to elicit sustained reversal of opioid elicited hypoxic brain injury, front brain region impairments (e.g., decreased memory, attention, spatial planning, and executive brain function), and/or increases in nitrotyrosine (NT) while causing sedation and analgesia. For example, the amount or therapeutically effective amount of the composition including the thiol-based compound described herein that is administered to the subject can be an amount effective to decrease hypoxic brain injury, decrease front brain region impairments, increase memory, increase attention, increase spatial planning, increase executive brain function, and/or decrease nitrotyrosine in the subject. For example, a brain injury and/or an opioid upon administration, (e.g., chronic opioid administration) to a subject can depress memory, attention, spatial planning, and/or executive brain function and/or increase nitrotyrosine (NT) at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80% or more, and the thiol-based compound described herein can be administered to the subject at an amount effective to increase memory, attention, spatial planning, and/or executive brain function, and/or decrease nitrotyrosine (NT) at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80% or more.

As will be appreciated by those of ordinary skill in the art, levels of nitrotyrosine (NT) in a subject correlate with oxidative nitric oxide damage and nitrosative stress related to brain injury, (e.g., an opioid related brain injury). It is contemplated that NT levels can be used as an in vivo marker of brain injury in a subject to indicate the presence and/or severity of brain injury in a subject. Therefore, in some embodiments, the method of treating a brain injury can further include the step of detecting a level of nitrotyrosine (NT) in a biological sample obtained from the subject.

In such methods, the NT levels determined for a biological sample obtained from the subject are compared to the level(s) in one or more control samples. The control samples can be obtained from a healthy individual (or a group of healthy individuals), from an individual (or group of individuals) afflicted with a brain injury, and/or from an individual (or group of individuals) afflicted with a specific severity of brain injury (e.g., mild, moderate, or severe brain injury as assessed using the Glasgow Coma Scale). The control level of NT can preferably be determined from a significant number of individuals, and an average or mean is obtained.

In certain embodiments, the control sample(s) are from a healthy individual (or group of individuals) and an increase of the level of NT in the subject's sample compared to the control value is indicative of the subject having a brain injury. Alternatively, when the level of NT reaches a sufficiently high level in one or more samples obtained from such subject, the detected level of NT can signal that the subject is at an increased probability for having a brain injury in comparison to a similar subject exhibiting a lower marker level and/or that a pharmaceutical composition described herein can be administered to the subject for the effective treatment of brain injury in the subject in accordance with the inventive method. In another embodiment, multiple determinations of the level of NT can be made, and a temporal change in the NT level can be used to monitor the efficacy of thiol-based compound compositions therapies. In such an embodiment, one might expect to see a decrease in the NT level over time during the course of effective therapy.

In some embodiments, the biological sample is obtained from a subject suspected of having a brain injury. In certain embodiments, the biological sample can be obtained from a subject suspected of having an opioid induced brain injury, such as a brain injury resulting from chronic or prolonged therapeutic opioid administration. In other embodiments, the biological sample is obtained from a subject suspected of having a brain injury not related to opioid use/abuse or therapeutic opioid administration (i.e., a non-opioid induced brin injury).

A biological sample obtained from the subject can include any type of biological sample allowing the level of NT in the subject to be assayed and correlated to brain injury. In some embodiments, the biological sample obtained from the subject is selected from the group consisting of cerebral spinal fluid (CSF), saliva, and blood using well known methods.

In certain embodiments, the biological sample obtained from a subject is a CSF sample. It has been shown that oxidative stress contributes to secondary brain injury in patients with traumatic brain injury (TBI) and poor neurologic outcome is associated with increased levels of NT in the CSF.

In some embodiments, the level of NT in a sample can be determined using one or more techniques including, but not limited to, MS-based methods (e.g., LC/MS/MS, GC/MS, and GC/MS/MS), HPLC, and immunological assay methods (e.g., ELISA). In certain embodiments, the level of NT in a biological sample is measured using an immunological assay. Assays for the measurement of NT are well known in the art.

In some embodiments, the biological sample can be collected from a subject and immediately analyzed to determine the level of nitrotyrosine (NT) in the sample. Alternatively, a processed sample can be stored, e.g., at −80° C. for analysis at a later time.

In some embodiments, compositions including a thiol-based compound described herein can be administered to the subject in combination with at least one additional compound, agent, and/or therapeutic agent useful for treating the subject, the breathing disorder, constipation and/or anuria, or the brain injury. These additional compounds, agents, and/or therapeutic agents can include commercially available agents or compounds, known to treat, prevent, or reduce the symptoms of breathing disorders or treat the disorder in the subject.

In some embodiments, the thiol-based compounds of formulas I, II, III, IV, V, VI, VII, VIII, or an adduct, a pharmaceutically acceptable salt, a tautomer, or a solvate thereof described herein can be administered to the subject in combination with at least one additional compound, agent, and/or therapeutic agent useful for treating the breathing disorder (e.g., opioid induced ventilatory and/or respiratory depression in the subject). In some embodiments, the at least one additional therapeutic agent can change normal breathing in a subject. Such additional agents can be selected from the group consisting of an opioid, doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists, ampakines, and combinations thereof.

In other embodiments, compositions comprising thiol-based compounds of formulas I, II, III, IV, V, VI, VII, VIII, or an adduct, a pharmaceutically acceptable salt, a tautomer, or a solvate thereof described herein and at least one additional compound has additive, complementary or synergistic effects in the treatment of the breathing disorder or other disorder in the subject. In a non-limiting example, the compositions that include a thiol-based compound described herein may be used concurrently or in combination with one or more of the following drugs: an opioid (e.g., morphine, oxycodone, fentanyl), doxapram, enantiomers of doxapram, acetazolamide, almitrine, theophylline, caffeine, methylprogesterone and related compounds, sedatives that decrease arousal threshold in sleep disordered breathing patients (e.g., eszopiclone and zolpidem), sodium oxybate, benzodiazepine receptor agonists (e.g., zolpidem, zaleplon, eszopiclone, estazolam, flurazepam, quazepam, temazepam, triazolam), orexin antagonists (e.g., suvorexant), tricyclic antidepressants (e.g., doxepin), serotonergic modulators, adenosine and adenosine receptor and nucleoside transporter modulators, cannabinoids (e.g., but not limited to, dronabinol), orexins, melatonin agonists (e.g., ramelteon) and compounds known as ampakines.

The combination of two or more compounds may refer to a composition wherein the individual compounds are physically mixed or wherein the individual compounds are physically separated. A combination therapy encompasses administering the components separately to produce the desired additive, complementary or synergistic effects.

In one embodiment, the composition comprising a thiol-based compound described herein and an additional agent are physically mixed in the composition. In another embodiment, the composition comprising a thiol-based compound described herein and the additional agent are physically separated in the composition.

In one embodiment, compositions including the thiol-based compound described herein are co-administered with a compound that is used to treat another disorder but causes loss of breathing control. In this aspect, compositions including the thiol-based compound described herein block or otherwise reduce depressive effects on normal breathing control caused by the compound with which they are co-administered. An exemplary compound that treats another disorder but depresses breathing control includes but is not limited to anesthetics, sedatives, sleeping aids, anxiolytics, hypnotics, alcohol, and narcotic analgesics. The co-administered compound may be administered individually, or a combined composition as a mixture of solids and/or liquids in a solid, gel or liquid formulation or as a solution, according to methods known to those familiar with the art.

In some embodiments, a composition including the thiol-based compound described herein may be packaged with at least one additional compound useful in the methods described herein. In another embodiment, a composition including a thiol-based compound described herein may be packaged with a therapeutic agent known to cause changes in breathing control, such as, but not limited to, anesthetics, sedatives, anxiolytics, hypnotics, alcohol, and narcotic analgesics. A co-package may be based upon, but not limited to, dosage units. For example, a composition can include an opioid capable of inducing ventilatory and/or respiratory depression in a subject and an amount of a thiol-based compound described herein effective to prevent the opioid induced ventilatory and/or respiratory depression when the composition is administered to the subject.

In yet another embodiment, the composition and the agent are separately administered to the subject. In yet another embodiment, the compound and the agent are co-administered to the subject.

Still other embodiments described herein relate to a composition that includes an opioid capable of inducing ventilatory and/or respiratory depression, inducing constipation and/or anuria, and/or inducing brain injury in a subject and an amount of thiol-based compound effective to attenuate the opioid induced ventilatory and/or respiratory depression, opioid induced constipation and/or anuria, and/or opioid induced brain injury when the composition is administered to the subject.

In some embodiments, the opioid can include at least one of alfentanil, buprenorphine, butorphanol, carfentanil, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanil, sufentanil, tapentadol, and tramadol, and pharmaceutically acceptable salts thereof. For example, the opioid can be carfentanil, fentanyl, remifentanil, or sufentanil.

In some embodiments, an effective amount (i.e., dose) of the thiol-based compound described herein (e.g., L-NAC) to be administered to a subject can be determined depending upon, for example, age, body weight, symptom, the desired therapeutic effect, the route of administration, and the duration of the treatment. Exemplary doses can be from about 0.01 to about 1000 mg, by oral administration. Examples of dose ranges can include from a minimum dose of about 0.01, 0.10, 0.50, 1, 5, 10, 25, 50, 100, 125, 150, 200, or 250 mg to a maximum dose of about 300, 400, 500, 600, 700, 800, 900, or 1000 mg, wherein the dose range can include from any one of the foregoing minimum doses to any one of the foregoing maximum doses. Specific examples of particular effective amounts contemplated via oral or intranasal administration can include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 mg or more. The oral dose can be administered once daily, twice daily, three times daily, or more frequently.

The dose of the thiol-based compound (e.g., L-NAC) for use in parenteral administration (e.g., intravenous administration) is generally from about 0.01 to about 300 mg/kg body weight. Examples of dose ranges can include from a minimum dose of about 0.01, 0.10, 0.50, 1, 5, 10, 25, 50, or 100 mg/kg body weight to a maximum dose of about 125, 150, 175, 200, 250, 275, or 300 mg/kg body weight, wherein the dose range can include from any one of the foregoing minimum doses to any one of the foregoing maximum doses. Specific examples of effective amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 mg/kg body weight or more. Continuous intravenous administration is also contemplated for from 1 to 24 hours per day to achieve a target concentration from about 0.01 mg/L blood to about 100 mg/L blood. Exemplary dose ranges can include from a minimum dose of about 0.01, 0.10, 0.25, 0.50, 1, 5, 10, or 25 mg/L blood to a maximum dose of about 40, 45, 50, 55, 60, 65, 70, 75, 80, 85, 90, or 100 mg/L, wherein an exemplary dose ranges can include from any one of the foregoing minimum doses to any one of the foregoing maximum doses. Specific examples of particular effective amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg/L blood or more. The dose to be used can depend upon various conditions, and there may be cases wherein doses lower than or greater than the ranges specified above are used.

The thiol-based compounds described herein (e.g., L-NAC) may be administered in the form of, for example, solid compositions, liquid compositions, or other compositions for oral administration, intranasal administration (e.g., dorsonasally), injections, liniments, or suppositories for parenteral administration. Solid compositions for oral administration include compressed tablets, pills, capsules, dispersible powders, and granules. Capsules include hard capsules and soft capsules. In such solid compositions, the stable L-NAC can be admixed with an excipient (e.g., lactose, mannitol, glucose, microcrystalline cellulose, or starch), combining agents (e.g., hydroxypropyl cellulose, polyvinyl pyrrolidone, or magnesium metasilicate aluminate), disintegrating agents (e.g., cellulose calcium glycolate), lubricating agents (e.g., magnesium stearate), stabilizing agents, agents to assist dissolution (e.g., glutamic acid or aspartic acid), or the like. The agents may, if desired, be coated with coating agents (e.g., sugar, gelatin, hydroxypropyl cellulose, or hydroxypropylmethyl cellulose phthalate), or be coated with two or more films. Further, coating may include containment within capsules of absorbable materials such as gelatin.

Liquid compositions for oral and/or intranasal administration include pharmaceutically acceptable solutions, suspensions, emulsions, syrups, and elixirs. In such compositions, the thiol-based compound is dissolved, suspended, or emulsified in a commonly used diluent (e.g., purified water, ethanol, or mixture thereof). Furthermore, such liquid compositions may also comprise wetting agents, suspending agents, emulsifying agents, flavoring agents (e.g., flavor-masking agents) sweetening agents, perfuming agents, preserving agents, buffer agents, or the like.

The thiol-based compounds of formulas I, II, III, IV, V, VI, VII, VIII, or an adduct, a pharmaceutically acceptable salt, a tautomer, or a solvate thereof described herein may be administered in the form of compositions for oral and/or intranasal administration via inhalation. In some embodiments, compositions for oral and/or intranasal administration via inhalation can be formulated with a mucosal penetration enhancer, i.e., a reagent that increases the rate or facility of transmucosal penetration of the compounds, such as but not limited to, a bile salt, fatty acid, surfactant or alcohol. In specific embodiments, the permeation enhancer can be sodium cholate, sodium dodecyl sulphate, sodium deoxycholate, taurodeoxycholate, sodium glycocholate, dimethylsulfoxide or ethanol.

In some embodiments, the thiol-based compounds described herein may be administered in the form of compositions for intranasal administration. The intranasal route of administration can effectively deliver therapeutic compounds into the brain of the subject where the compounds bypass the blood-brain barrier (BBB) and enter the brain directly through the olfactory route. Intranasal administration can include inserting an intranasal drug delivery device (e.g., an inhaler) into a nostril of the patient, urging the device through the nostril such that the distal end of the body is in the apex of the nasal cavity, and contacting a portion of the nasal epithelium (e.g., a portion overlying the sphenopalatine ganglion (SPG)) with the composition.

Administration can include administration though a delivery device. For example, the delivery device is intended to encompass an inhaler (e.g., a propellant driven inhaler), nebulizer, atomizer, pump aerosolizer of a liquid formulation, and aerosolizer of a dry powder formulation. As used herein, the term “inhaler” refers both to devices for nasal and pulmonary administration of a drug, e.g., in solution, powder and the like. In some embodiments, the inhaler drug delivery device can include a propellant driven inhaler or plastic spray bottles. In certain embodiments, the delivery device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art.

For nasal administration, a useful device is a small, hard bottle to which a metered dose sprayer is attached. In one embodiment, the metered dose is delivered by drawing a pharmaceutical composition described herein into a chamber of defined volume, which chamber has an aperture dimensioned to aerosolize and aerosol formulation by forming a spray when a liquid in the chamber is compressed. The chamber is compressed to administer the composition. In a specific embodiment, the chamber is a piston arrangement. Such devices are commercially available.

In an alternative embodiment, a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation. Preferably, the sprayer delivery device will provide a metered amount of the aerosol formulation, for administration of a measured dose of the thiol-based compound.

Often, the aerosolization of a liquid or a dry powder formulation for inhalation into the lung will require a propellent. The propellent may be any propellant generally used in the art. Specific nonlimiting examples of such useful propellants are a chloroflourocarbon, a hydrofluorocarbon, a hydochlorofluorocarbon, or a hydrocarbon, including trifluoromethane, dichlorodiflouromethane, dichlorotetrafluoroethanol, and 1,1,1,2-tetraflouroethane, or combinations thereof.

In some embodiments, a dry powder formulation for inhalation includes a finely divided powder form of a thiol-based compound described herein and a dispersant. For example, the dry powder formulation can include a finely divided dry powder containing a thiol-based compound described herein, a dispersing agent and also a bulking agent. Bulking agents useful in conjunction with the present formulation include such agents as lactose, sorbitol, sucrose, or mannitol, in amounts that facilitate the dispersal of the powder from the device.

In some embodiments, the thiol-based compounds described herein may be administered in the form of compositions for nebulization administration. Similar to the inhalation route, thiol-based compound compositions given by nebulization must be aerosolized into small particles to reach the lungs. Nebulization requires the use of special devices, most commonly ultrasonic or jet nebulizer systems. Using the devices properly helps maximize the amount of drug delivered to the lungs.

Injections for parenteral administration include solutions, suspensions, emulsions, and solids, which are dissolved or suspended. For injections, the thiol-based compound can be dissolved, suspended, and/or emulsified in a solvent. The solvents are, for example, distilled water for injection, physiological salt solution, vegetable oil, propylene glycol, polyethylene glycol, alcohol such as ethanol, or a mixture thereof. Moreover, the injections also can include stabilizing agents, agents to assist dissolution (e.g., glutamic acid, aspartic acid, or POLYSORBATE 80), suspending agents, emulsifying agents, soothing agents, buffer agents, preserving agents, etc. The compositions are sterilized in the final process or manufactured and prepared by sterile procedure. The compositions also can be manufactured in the form of sterile solid compositions, such as a freeze-dried composition, and can be sterilized or dissolved immediately before use in sterile distilled water for injection or some other solvent.

Other compositions for parenteral administration include liquids and ointments for external use, endermic liniments, compositions for inhalation, sprays, suppositories for rectal administration, and pessaries for vaginal administration, which compositions include a stable thiol-based compound and are administered by methods known in the art.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions is contemplated include, but are not limited to, humans and other primates, mammals including commercially relevant mammals such as cattle, pigs, horses, sheep, cats, and dogs.

The regimen of administration may affect what constitutes an effective amount. The therapeutic formulations may be administered to the patient either prior to, currently, or after administration of the opioid. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds employed in the pharmaceutical composition at levels lower than that required in order to achieve the desired therapeutic effect and gradually increase the dosage until the desired effect is achieved.

In some embodiments, the pharmaceutical composition is administered concurrently with opioid administration and/or up to about 10 minutes, up to about 20 minutes, up to about 30 minutes, up to about 40 minutes, up to about 50 minutes, up to about 60 minutes, up to about 70 minutes, up to about 80 minutes, up to about 90 minutes, up to about 100 minutes, up to about 110 minutes, or up to about 120 minutes before or after initiation of opioid administration.

In other embodiments, the composition can be administered to the subject at an amount effective to prevent the need for mechanical ventilation in subjects with acutely impaired ventilatory and/or respiratory drive because of an acute requirement for narcotic analgesia.

The invention is further illustrated by the following example, which is not intended to limit the scope of the claims.

### Example 1

We have begun to study the ability of our L,D-thiolester drugs to overcome the ventilatory depressant effects elicited by continuous intravenous infusions of fentanyl in unanesthetized rats. Such infusions are used in adult and pediatric human patients but their ability to provide pain relief is often compromised by their propensity to cause respiratory depression. We now report our unexpected findings that intravenous injections of L-NAC reverse the deleterious effects of continuous fentanyl infusion on ventilatory parameters and ABG chemistry whereas they did not affect the sedative or analgesic effects of the opioid. It therefore appears that continuous infusion of fentanyl somehow sets up a scenario that allows for L-NAC to modulate intracellular signaling cascades the mediated fentanyl-induced OIRD. Systemic/oral administration of L-NAC is approved for human use for a variety of conditions and our findings show that L-NAC could be used for potential reversal of OIRD elicited by the infusion of fentanyl in human subjects.

### Methods

### Permissions, Rats, and Surgical Procedures

All studies were carried out in strict accordance with the NIH Guide for Care and Use of Laboratory Animals (NIH Publication No. 80-23) revised in 1996, and in strict compliance with the ARRIVE (Animal Research: Reporting of In Vivo Experiments) guidelines. All protocols involving the use of rats were approved by the Animal Care and Use Committees of Galleon Pharmaceuticals, the University of Virginia Case and Case Western Reserve University. Adult male Sprague Dawley rats were purchased from Harlan Industries (Madison, WI, USA). After five days of recovery from transportation, the rats received femoral artery catheters and/or two jugular vein catheters under 2-3% isoflurane anesthesia (10-14, 58). One of the jugular catheters was to allow for continuous infusion of fentanyl whereas the other allowed for bolus injections of vehicle or L-NAC. The rats were given five days to recover from surgery before use in experiments. All femoral arterial catheters were flushed daily with a heparin solution (50 units of heparin in 0.1 M, pH 7.4 phosphate-buffered saline). The arterial catheters were flushed with 0.3 ml of phosphate-buffered saline (0.1 M, pH 7.4) 2-3 h before the start of the study. The pH of all of the stock solutions of vehicle and L-NAC were adjusted to pH of 7.2 with 0.125M NaOH. All studies were done in a quiet laboratory room with a relative humidity of 51±2% and room temperature of 21.4±0.2° C. The ventilatory and ABG chemistry studies and the antinociception experiments were performed in separate groups of rats so as to not compromise the ventilatory recordings. The plethysmography and antinociception recording sessions and arterial blood sampling studies (ABG assays) were done by an investigator who injected the opioid, vehicle, or L-NAC. The syringes with vehicle or L-NAC were filled by another investigator, such that the experimenter was blinded to the protocol. In addition, the data files resulting from each of experiment were collated and analyzed by another investigator in the group.

### Protocols for Whole Body Plethysmography Measurement of Ventilatory Parameters

Ventilatory parameters were recorded continuously in unrestrained freely-moving rats by a whole body plethysmography system (PLY3223; Data Sciences International, St. Paul, MN) as described previously (see 9-14). The directly recorded and derived parameters (see 61-64) are defined in Table 2 and FIG. 10. These parameters and their abbreviations are as follows: frequency of breathing (Freq), tidal volume (TV), minute ventilation (MV), inspiratory time (Ti), expiratory time (Te), Ti/Te, end inspiratory pause (EIP), end expiratory pause (EEP), peak inspiratory flow (PIF), peak expiratory flow (PEF), PIF/PEF, expiratory flow at 50% expired TV (EF₅₀), relaxation time (RT), inspiratory drive (TV/Ti), expiratory drive (TV/Te), apneic pause [(Te/RT)-1] and non-eupneic breathing index (NEBI). On the day of study, each rat was placed in an individual plethysmography chamber and given 60-75 min to acclimatize so that baseline (pre) ventilatory parameter values could be accurately defined. Two groups of rats (n=6 per group) received a continuous infusion of fentanyl (1 μg/kg/min, IV) and after 60 min and 90 min, one group of rats received bolus injections of vehicle (saline, IV) whereas the other received injections of L-NAC (500 mol/kg, IV). Ventilatory parameters were monitored for a further 30 min following the second injection of vehicle or L-NAC. The body weights of the two groups were similar to one another (see Table 3) and as such, ventilatory parameters related to volumes such as TV, PIF, PEF and EF₅₀ are presented without correcting for body weight. FinePointe (DSI) software constantly corrected digitized ventilatory values originating from the respiratory waveforms for alterations in chamber humidity and temperature. Corrections of ventilatory parameters for alterations in body temperature were not necessary because temperatures recorded in other groups of rats changed minimally during the study and because these changes were virtually identical in the two groups (see Table 4). Pressure changes associated with the respiratory waveforms were converted to volumes (e.g., TV, PIF, PEF, EF₅₀) employing the algorithms of Epstein and colleagues. Specifically, factoring in chamber humidity and temperature, cycle analyzers filtered the acquired signals, and FinePointe algorithms generated an array of box flow data that identified a waveform segment as an acceptable breath. From that data vector, the minimum and maximum values were determined. Flows at this point were “box flow” signals and from this array, minimum and maximum box flow values were determined and multiplied by a compensation factor provided by the selected algorithm thus producing TV, PIF, PEF and EF₅₀ values that were used to determine non-eupneic breathing events expressed as the non-eupneic breathing index (NEBI), reported as the percentage of non-eupneic breathing events per individual epoch. Apneic pause was determined by the formula, (Expiratory Time/Relaxation Time)−1.

### Protocols for Blood Gas Measurements and Determination of Arterial-Alveolar Gradient

ABG chemistry parameters (pH, pCO₂, pO₂, sO₂) and A-a gradients were recorded before (Pre), 60 min after continuous infusion of fentanyl (1 μg/kg/min, IV), 30 min after an injection vehicle (n=9 rats, 83.1±0.4 days of age; 337±3 g) or L-NAC (500 mol/kg, IV; 83.5±0.3 days; 340±3 g), and again 30 min after a second injection of vehicle or L-NAC (500 μmol/kg, IV) as previously described. The samples of arterial blood (100 μL) taken at the above times were injected into a Radiometer blood-gas analyzer (ABL800 FLEX) to obtain ABG values. The A-a gradient defines differences between alveolar O₂ and arterial blood O₂ concentrations. A reduction in PaO₂, without a concomitant alteration in A-a gradient is due to hypo-ventilation, whereas a decrease in PaO₂ with a concomitant elevation in A-a gradient indicates an on-going mismatch in ventilation-perfusion in alveoli. A-a gradient=PAO₂−PaO₂, where PAO₂ is the partial pressure (p) of alveolar O₂ and PaO₂ is pO₂ in the sampled arterial blood. PAO₂=[(FiO₂×(Pₐₜₘ−P_{H2O})−(PaCO₂/respiratory quotient)], where FiO₂ is the fraction of O₂ in inspired air; Pₐₜₘ is atmospheric pressure; P_{H2O} is the partial pressure of H₂O in inspired air; PaCO₂ is pCO₂ in arterial blood; and respiratory quotient (RQ) is the ratio of CO₂ eliminated/O₂ consumed. We took FiO₂ of room-air to be 21%=0.21, Pₐₜₘ to be 760 mmHg, and P_{H2O} to be 47 mmHg (Gaston et al., 2021). We took the RQ value of our adult male rats to be 0.9.

### Antinociception Assessment by Tail-Flick Latency Assay

We recorded TFL values before (Pre), 30 and 60 min during continuous infusion of fentanyl (1 μg/kg/min, IV), 15 and 30 min after an injection vehicle (n=9 rats, 82.2±0.3 days of age; 331±3 g) or L-NAC (500 μmol/kg, IV; 82.7±0.4 days; 333±3 g), and again 15 and 30 min after a second injection of vehicle or L-NAC (500 mol/kg, IV) via the use of a Tail-Flick Analgesia Meter (IITC Life Science Inc., USA) as detailed previously. The procedure involved a minor degree of manual restraint while positioning the tail to apply a thermal beam sufficient to induce a latency of tail withdrawal of approximately 2.5 sec. TFL data are shown as actual TFL (sec) and as maximum possible effect (% MPE) determined by the formula, % MPE=[(post-injection TFL−baseline TFL)/(12−baseline TFL)]×100 (10−14, 58).

### Body Temperatures, and Sedation as Determined by the Modified Righting Reflex Test

We recorded rat body temperatures before (Pre), 30 and 60 min during continuous infusion of fentanyl (1 μg/kg/min, IV), 15 and 30 min after an injection vehicle (n=9 rats, 83.4±0.4 days of age; 336±3 g) or L-NAC (500 μmol/kg, IV; 83.9±0.4 days; 339±3 g), and again 15 and 30 min after a second injection of vehicle or L-NAC (500 mol/kg, IV) via the use of a rectal thermometer (DSI Inc., USA) as detailed previously. The rats were placed in separate open plastic boxes and allowed 45-60 min to acclimatize. A thermistor probe was inserted 5-6 cm into the rectum to allow for regular recordings of body temperature. A 2-3 inch length of the probe cable connected to a telethermometer (YellowSprings Instruments, South Burlington, Vermont), was taped to the tail. These and all rats used in the other studies were not fasted prior to use. The ability of rats to regain their feet after being placed on their side was also determined following each body temperature measurement.

### Data Analyses

The directly recorded and arithmetically-derived ventilatory parameters (1 min bins) were taken for statistical analyses. Pre-drug 1 min bins excluded occasional marked deviations from resting values due to abrupt movements by the rats such as scratching. The exclusions ensured accurate assessment of baseline values. All ventilatory, ABG chemistry, A-a gradient, TFL, body temperature and righting reflex data are presented as mean±SEM and were evaluated using one-way and two-way ANOVA followed by Bonferroni corrections for multiple comparisons between means using the error mean square terms from each ANOVA analysis as detailed previously. A P<0.05 value denoted the initial level of statistical significance that was modified according to the number of comparisons between means as described by Wallenstein et al. The modified t-statistic is t=(mean group 1−mean group 2)/[s×(1/n₁+1/n₂)^{1/2}] where s²=the mean square within groups term from the ANOVA (the square root of this value is used in the modified t-statistic formula) and n₁ and n₂ are the number of rats in each group under comparison. Based on an elementary inequality called Bonferroni's inequality, a conservative critical value for modified t-statistics obtained from tables of t-distribution using a significance level of P/m, where m is the number of comparisons between groups to be performed. The degrees of freedom are those for the mean square for within group variation from the ANOVA table. In most situations, the critical Bonferroni value cannot be found in conventional tables of the t-distribution but can be approximated from tables of the normal curve by t*=z+(z+z³)/4n, with n being the degrees of freedom and z being the critical normal curve value for P/m. Wallenstein et al first demonstrated that the Bonferroni procedure is preferable for general use since it is easy to apply, has the widest range of applications, and because it provides critical values that are lower than those of other procedures when the investigator can limit the number of comparisons (and will be slightly larger than those of other procedures if many comparisons are made. As mentioned, a value of P<0.05 was taken as the initial level of statistical significance and statistical analyses were performed with the aid of GraphPad Prism software (GraphPad Software, Inc., La Jolla, CA).

### Results

### Ventilatory Parameters

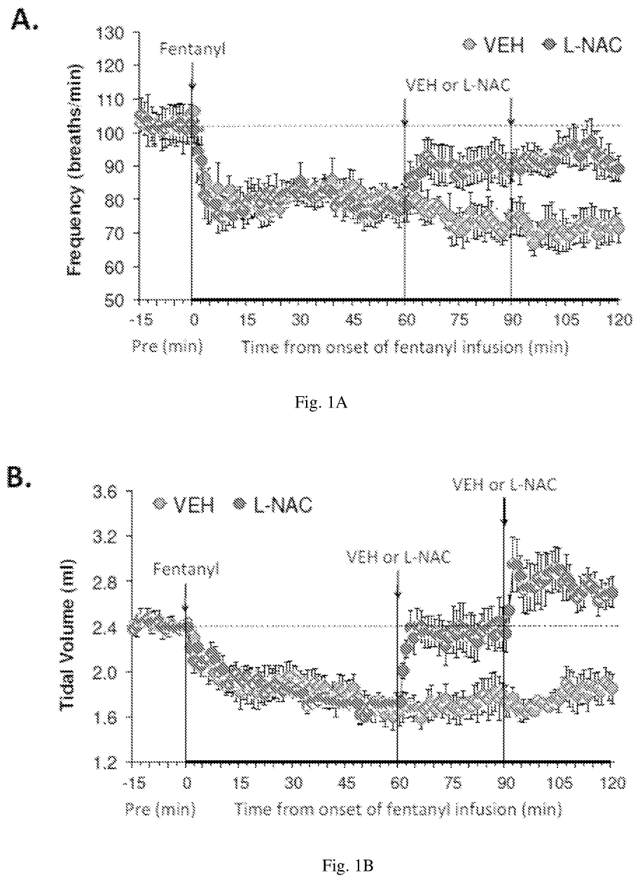

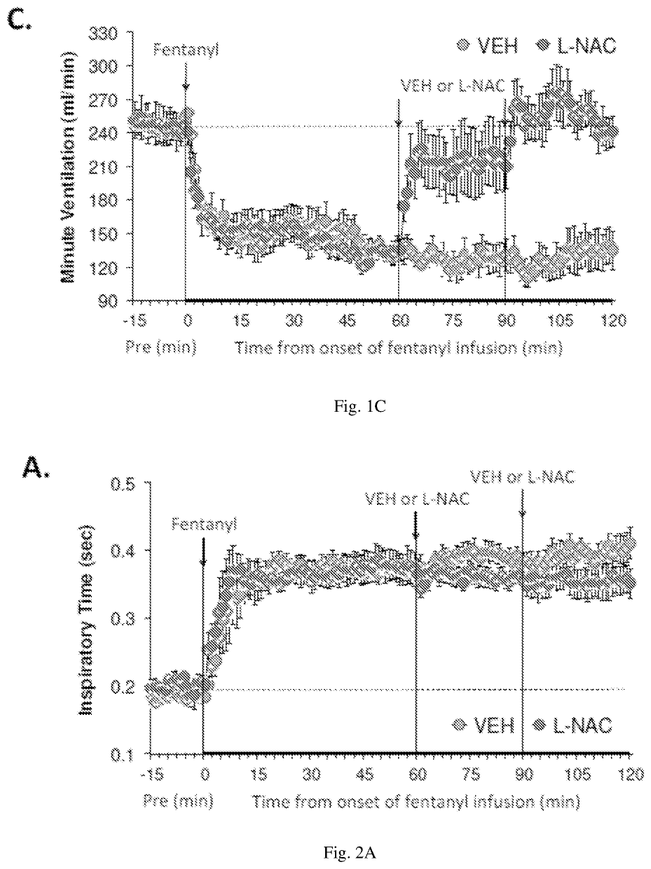

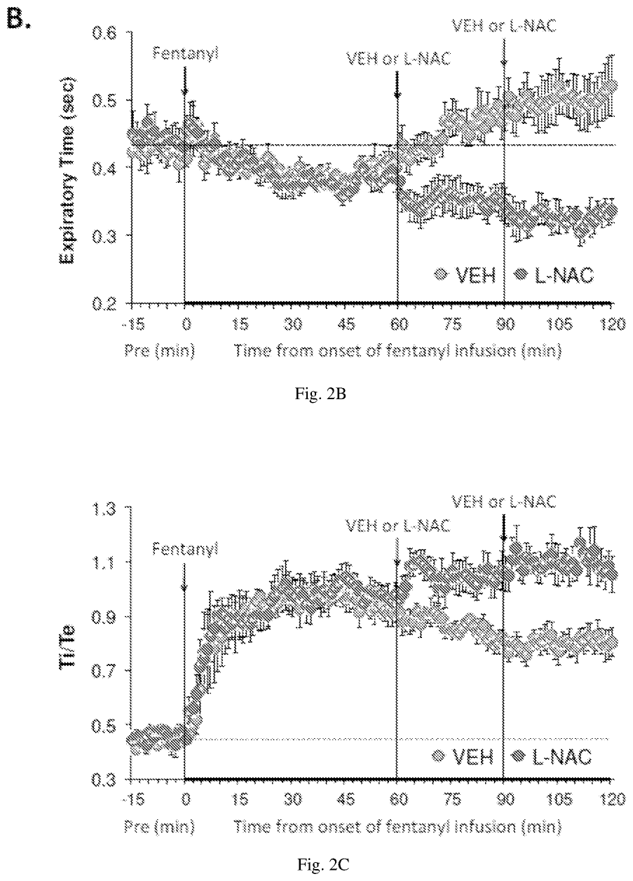

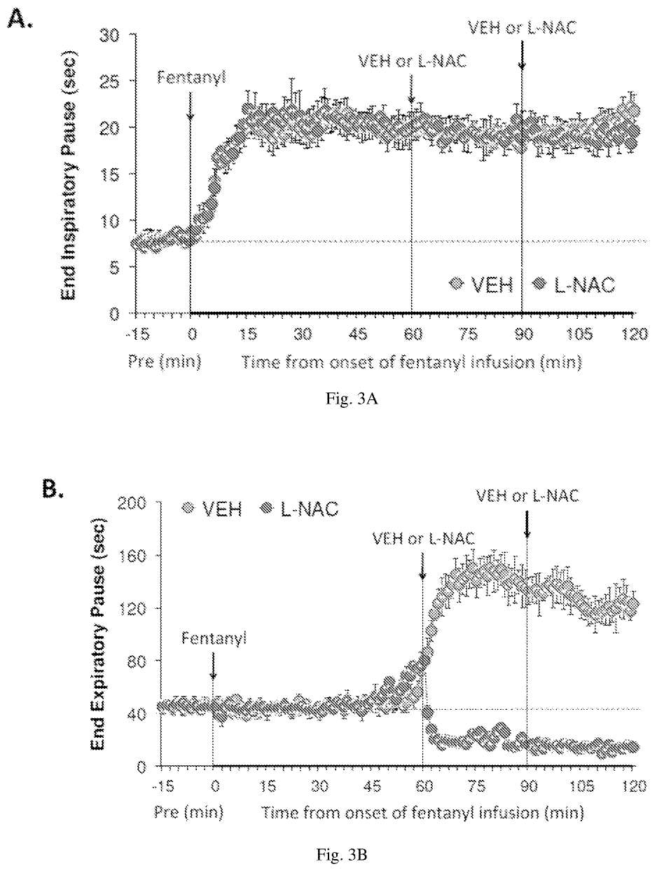

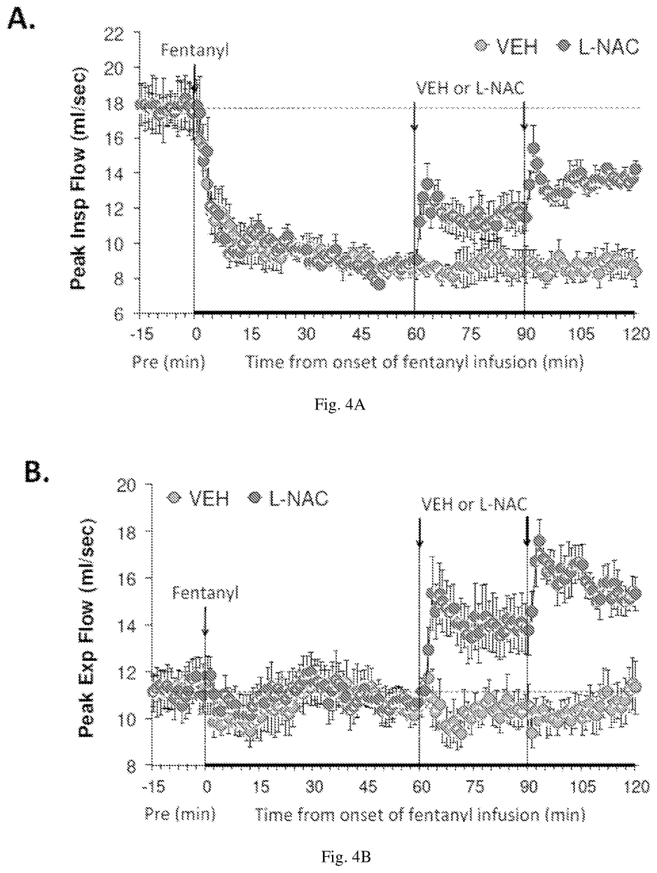

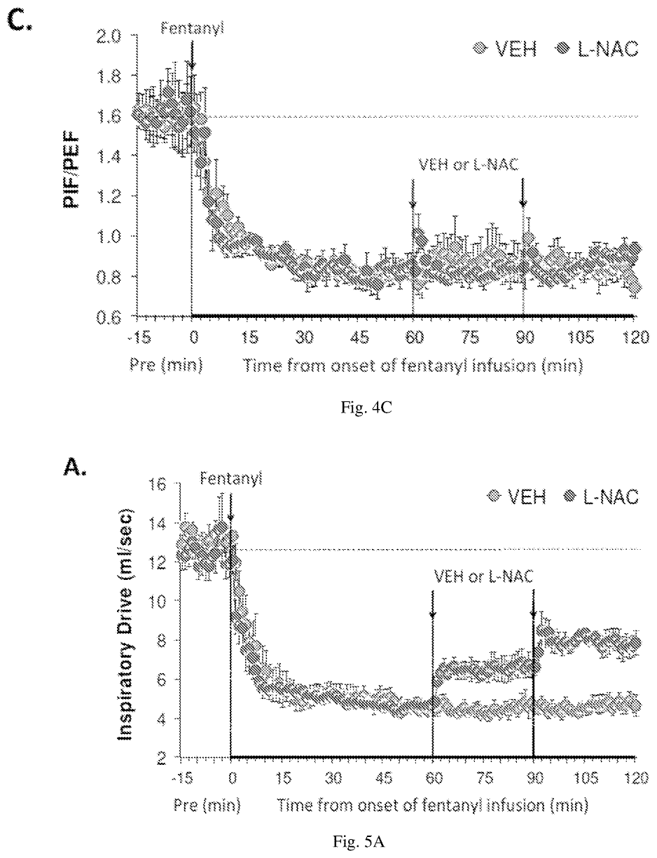

Descriptions of the ventilatory parameters used in this study are provided in Table 2 and FIG. 10. There were no differences in baseline (pre-fentanyl) parameters between the two groups of rats (Table 3). It is important to note that the infusion of fentanyl (1 g/kg/min, IV) and subsequent injection of vehicle or L-NAC (500 mol/kg, IV) exerted minor changes in body temperature (Table 4) that were therefore not a factor in the expression of the ventilatory responses described below (See Methods for explanation). Actual values of frequency of breathing (Freq), tidal volume (TV) and minute ventilation (MV) before (Pre) and during the infusion of fentanyl (1 μg/kg/min, IV) and subsequent injections of vehicle (VEH) or L-NAC (500 mol/kg, IV) given at the 60 and 90 min infusion time-points are shown in FIG. 1. The infusion of fentanyl elicited pronounced decreases in Freq, TV and MV that were equivalent in the two groups. The two injections of vehicle elicited minor immediate responses and presumably did not affect the temporal effects of fentanyl infusion. In contrast, the first injection of L-NAC (500 mol/kg, IV) caused immediate and sustained increase in these parameters to levels approaching (Freq and MV) and actually reaching (TV) pre-infusion levels. The second injection of L-NAC did not further boost Freq whereas it did boost TV and therefore MV. Inspiratory time (Ti), expiratory time (Te) and Ti/Te (inspiratory quotient) during various stages of the study are summarized in FIG. 2. The infusion of fentanyl elicited a substantial and sustained increases in Ti that was only minimally affected by injections of vehicle or L-NAC (Panel A). As seen in Panel B, the infusion of fentanyl elicited a relatively minor decrease in Te in the group that was to receive the injections of vehicle that beginning at about 45-50 min turned to an increase in Te that was sustained at the latter stages of infusion. The L-NAC injections prevented this increase in Te such that the slow downward trajectory of Te during infusion was sustained. As such, the injections of L-NAC caused a sustained elevation in Ti/Te compared to vehicle-injected rats (Panel C). End inspiratory pause (EIP), and expiratory pause (EEP) during various stages of the study are summarized in FIG. 3. Peak inspiratory flow (PIF), peak expiratory flow (PEF) and PIF/PEF (flow balance) during various stages of the experiment are shown in FIG. 4. The fentanyl infusion elicited a pronounced and sustained decrease in PIF that was unaffected by injections of vehicle whereas the injections of L-NAC elicited a sustained partial recover of PIF (Panel A). The infusion of fentanyl elicited minor effects on PEF. The injections of L-NAC elicited substantial and sustained increases in PEF (Panel B). As a result, the substantial decrease in PIF/PEF during infusion of fentanyl was not affected by the injections of vehicle or L-NAC (Panel C).

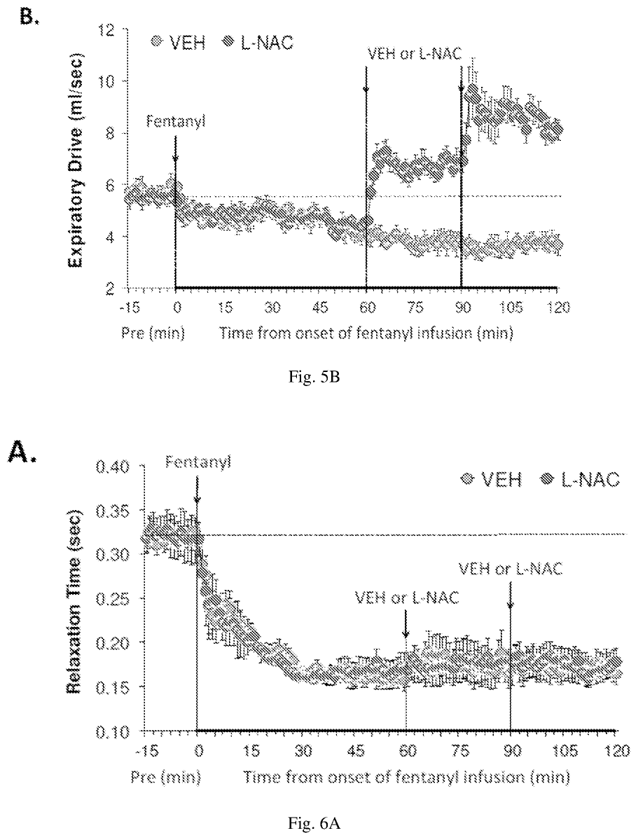

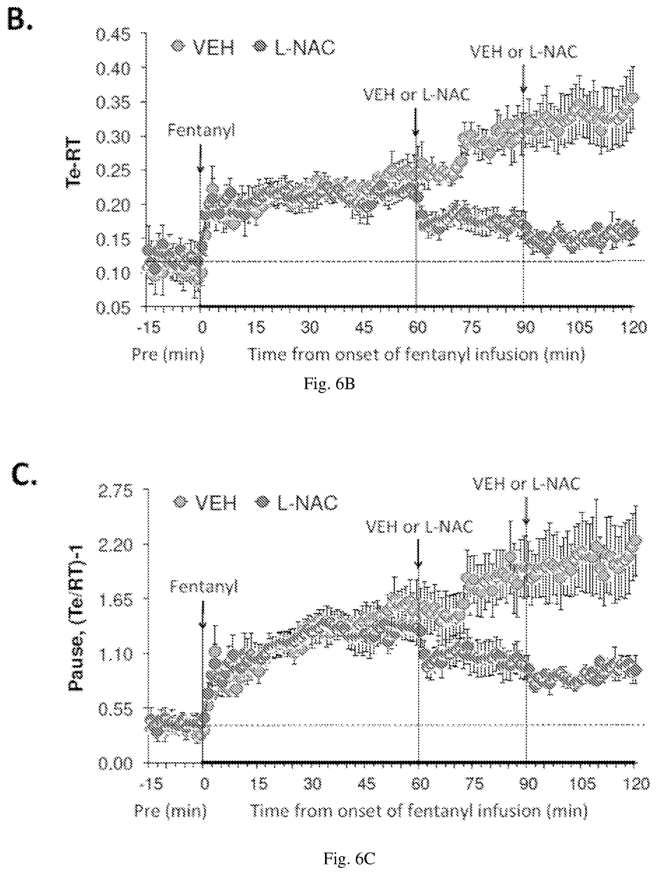

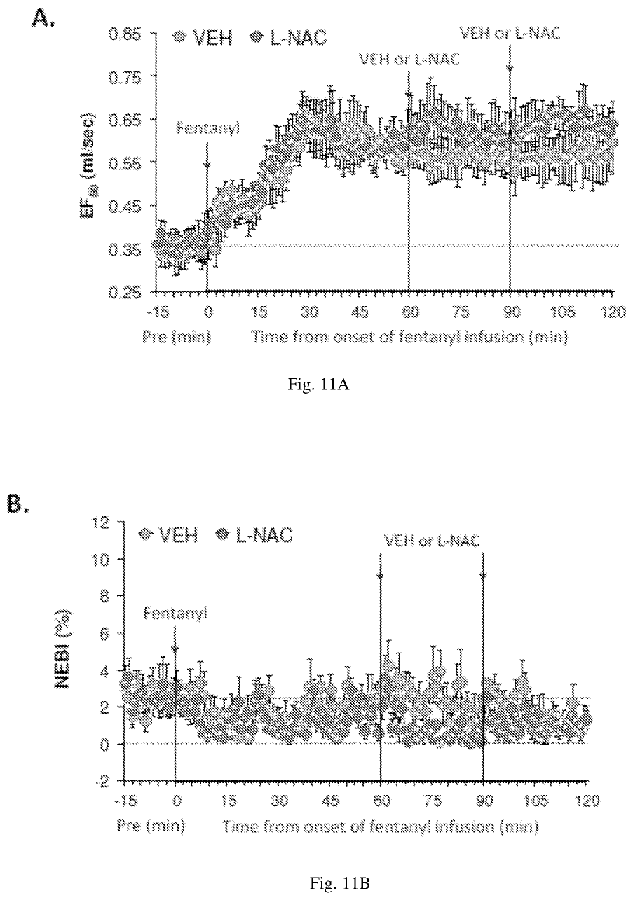

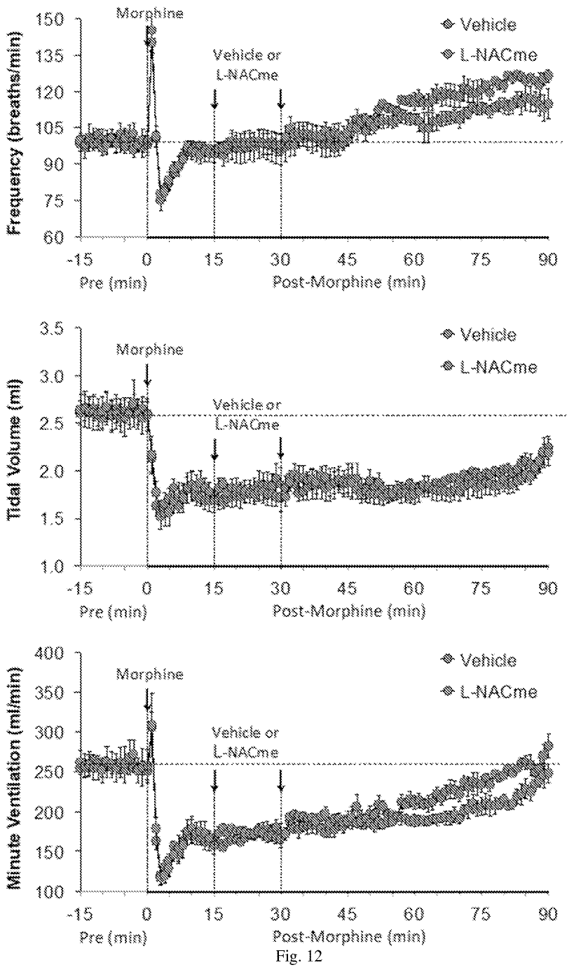

Inspiratory (TV/Ti) and expiratory (TV/Te) drives during various stages of the experiment are shown in FIG. 5. The infusion of fentanyl elicited a pronounced and sustained decreases in inspiratory drive that was partially reversed by injections of L-NAC (Panel A). The fentanyl infusion elicited gradual and eventually substantial decreases in expiratory drive in the rats that received injections of vehicle whereas the injections of L-NAC elicited immediate, substantial and sustained increases in expiratory dive (Panel B). Relaxation time (RT), expiratory delay (Te-RT) and expiratory ratio [(Te/RT)−1] during various stages of the experiment are shown in FIG. 6. The infusion of fentanyl elicited a substantial and sustained decrease in RT that was not affected by the injections of L-NAC (Panel A). The infusion of fentanyl also caused a sustained increase in expiratory delay (Panel B) and expiratory ratio (Panel C) that was markedly diminished by the injections of L-NAC. Expiratory flow at 50% tidal volume (EF₅₀) and non-eupneic breathing index (NEBI) during various stages of the study are shown in FIG. 11. The fentanyl infusion elicited substantial increases in EF₅₀ (Panel A) that were only minimally affected by L-NAC. The fentanyl infusion did not clearly affect the low level of non-eupneic breathing (Panel B). The first L-NAC injection caused a noticeable decrease in NEBI whereas the level of NEBI after the second injection of L-NAC was not noticeably different from those that received the second injection of vehicle.

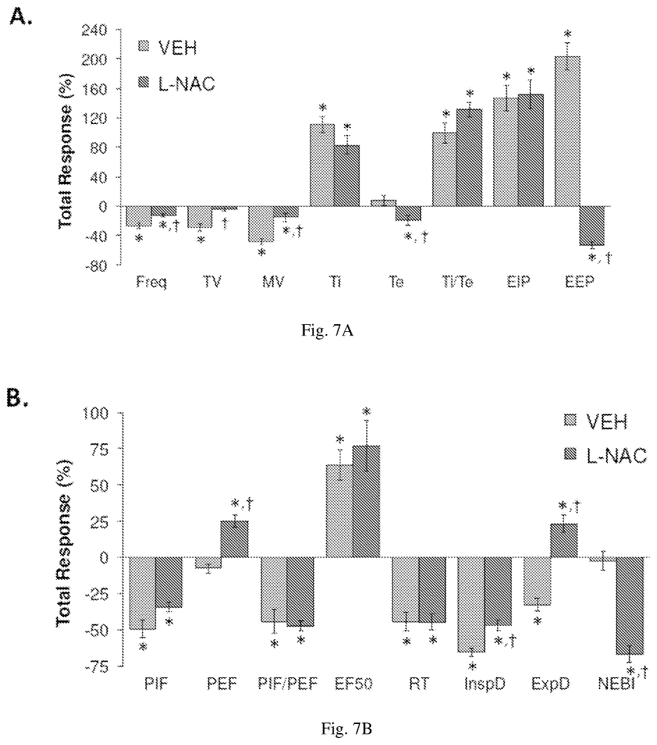

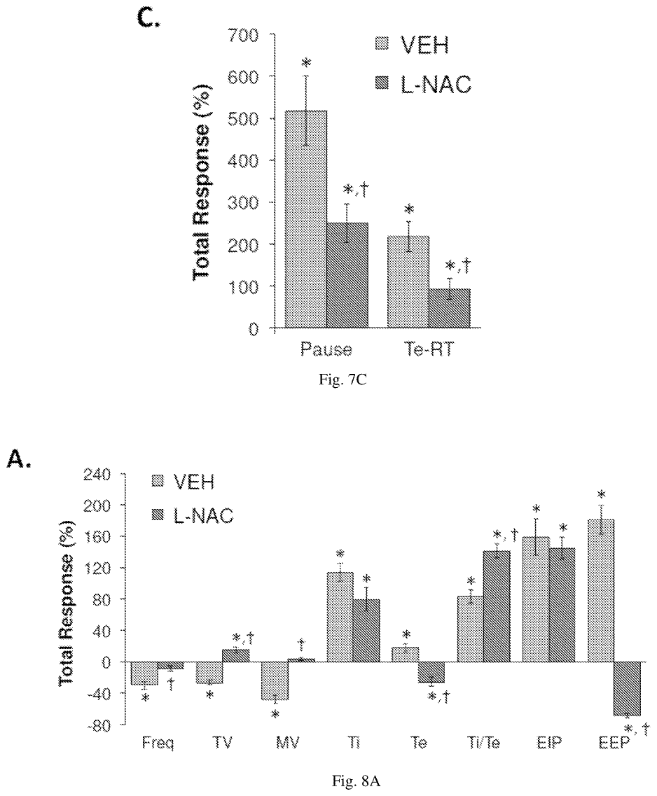

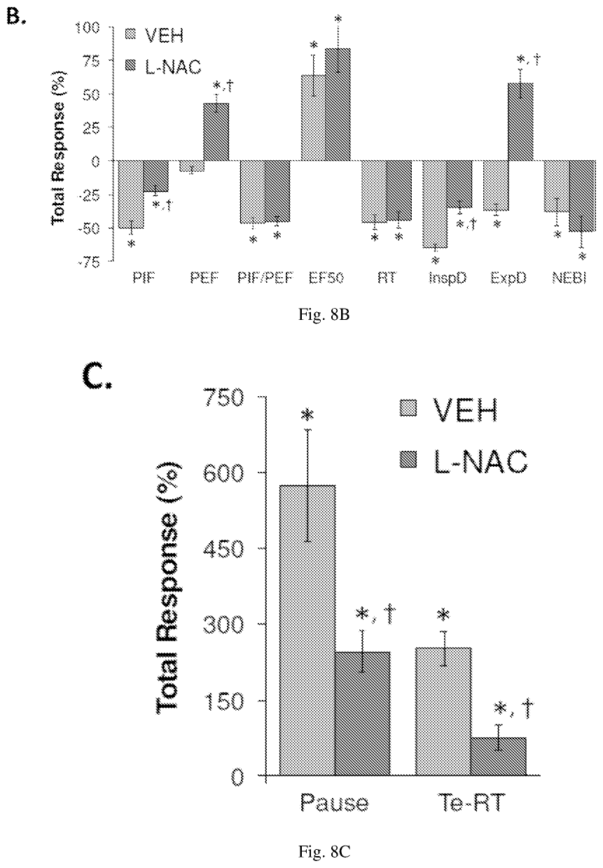

A summary of the total (cumulative changes in ventilatory parameters recorded over the first 60 min of fentanyl infusion (1 μg/kg/min, IV) in the two experimental groups of summarized in Table 5. As described above, the infusion of fentanyl elicited pronounced decreases in Freq, TV, MV, PIF, PIF/PEF, RT, inspiratory drive, expiratory drive and NEBI, substantial increases in Ti, Ti/Te, EIP, EF50, apneic pause and RT-Te but minimal changes in Te, EEP or PEF. Ventilatory parameters at key stages of the studies are shown in Tables 6 and 7. The stages are pre-values, values at 60 min of infusion (i.e., immediately prior to the first injection of vehicle or L-NAC values at 90 min of infusion (30 min post-injection one and immediately prior to injection two) and at 120 min of infusion (30 min post injection two). These tables provide summary data and statistics to support the conclusions above concerning the effects of the infusion of fentanyl on baseline ventilatory parameters and the responses by the two injections of vehicle or L-NAC. A summary of the total (cumulative) changes in ventilatory parameters elicited by the first injection of vehicle or L-NAC (500 mol/kg, IV) in rats receiving an infusion of fentanyl (1 μg/kg/min, IV) are summarized in FIG. 7. These values are expressed as % changes from pre-fentanyl values so as to determine the degree of reversal by L-NAC. As seen in Panel A, the first L-NAC injection reversed the adverse effects of fentanyl on Freq, TV and MV and EEP but did not affect the fentanyl-induced increases in Ti, Ti/Te or EEP. As seen in Panel B, the first injection of L-NAC only partially reversed the fentanyl-induced decrease in inspiratory drive (InspD) whereas it converted the decrease in expiratory drive (ExpD) to an increase. L-NAC elicited a pronounced increase in PEF and virtually eliminated NEBI but did not affect the fentanyl-induced changes in PIF, PIF/PEF and RT. As can be seen in Panel C, the injection of L-NAC caused a marked reduction in the fentanyl-induced increase in apneic pause and relative expiratory delay (Te-RT). A summary of the total (cumulative) changes in ventilatory parameters elicited by injection two of vehicle or L-NAC (500 μmol/kg, IV) in the rats receiving infusion of fentanyl (1 μg/kg/min, IV) are summarized in FIG. 8 (values expressed as % changes of pre-fentanyl values). As seen in Panel A, the second injection of L-NAC reversed the deleterious effects of fentanyl on Freq, TV, MV, Te and EEP, but did not affect the fentanyl-induced changes in Ti, Ti/Te or EIP. As seen in Panel B, the second injection of L-NAC reversed the effects of fentanyl on PIF, PEF (but not PIF/PEF) and inspiratory (InspD) and expiratory (ExpD) drives but not EF50, RT or NEBI. As seen in Panel C, the second injection of L-NAC caused a substantial reduction in the fentanyl-induced increase in apneic pause and relative expiratory delay (Te-RT).

### Arterial Blood Gas Chemistry

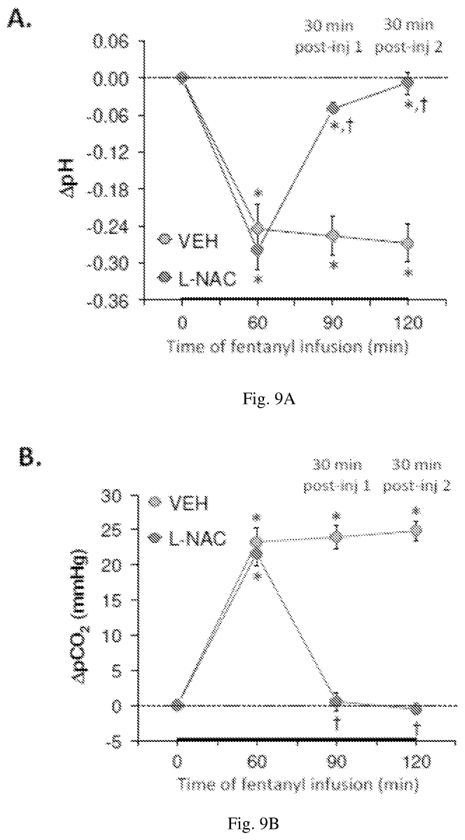

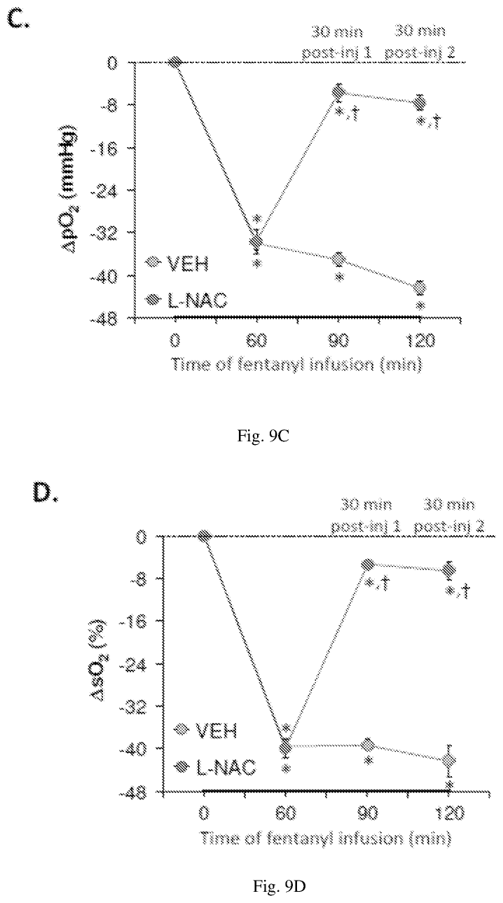

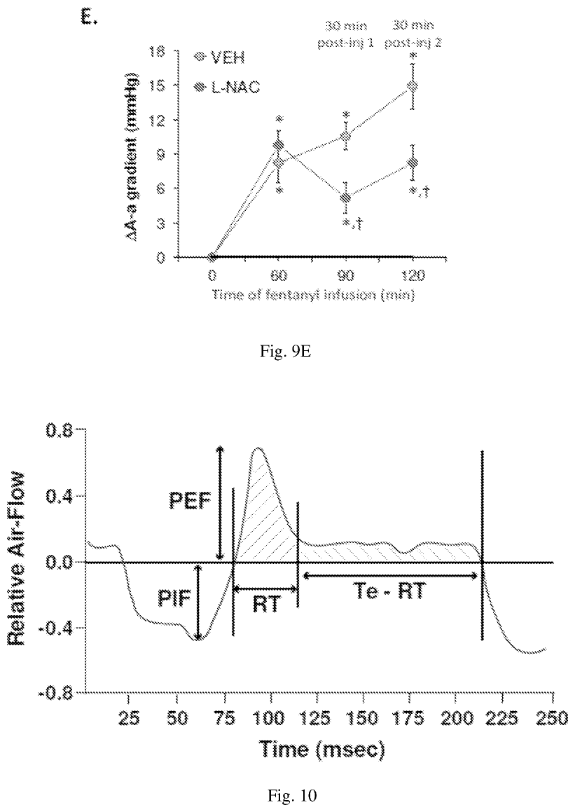

The values for pH, pCO₂, pO₂, sO₂ and A-a gradient at various stages of the experiment in the two groups of rats are summarized Table 8. The arithmetic changes in these parameters that occurred at 60 min time-point of infusion of fentanyl (1 μg/kg/min, IV) in the two groups of rats, 30 min after the first injection of vehicle or L-NAC (500 mol/kg, IV) and 30 min after the second injection of vehicle or L-NAC (500 mol/kg, IV), are summarized in FIG. 9. The infusion of fentanyl elicited profound decreases in pH, pO₂ and sO₂ after 60 min of infusion that were accompanied by equally pronounced increases in pCO₂ and A-a gradient. The two injections of vehicle given 30 min apart did not elicit appreciable changes in these values. In contrast, the first injection of L-NAC elicited a pronounced reversal of the negative effects of fentanyl on all ABG chemistry and A-a gradient values as recorded 30 min after injection. The beneficial effects of L-NAC were still evident 30 min after the second injection of L-NAC.

### Tail-Flick Latencies

T L values recorded at various stages of the experiments are shown in Table 1. As can be seen, the infusion of fentanyl (1 μg/kg/min, IV) elicited a sustained increase in TFL in the two groups of rats that was evident within 15 min. The injections of vehicle or L-NAC (500 μmol/kg, IV) did not affect the antinociceptive effects of the fentanyl infusion.

TABLE 1
Antinociceptive status during various stages of the experiments

| | | Injection | Study Group | |
|---|---|---|---|---|
| Parameters | Study Phase | Phase | Vehicle | L-NAC |
| Actual TFL | Pre | | 2.2 ± 0.1 | 2.1 ± 0.1 |
| values, sec | | | | |
| | Post-infusion | | 11.6 ± 0.2 | 11.5 ± 0.1 |
| | 15 min | | | |
| | Post-infusion | | 11.9 ± 0.1 | 12.0 ± 0.0 |
| | 30 min | | | |
| | Post-infusion | | 11.9 ± 0.1 | 11.9 ± 0.1 |
| | 45 min | | | |
| | Post-infusion | | 12 ± 0.0 | 11.9 ± 0.1 |
| | 60 min | | | |
| | Post-infusion | Post-drug | 12 ± 0.0 | 11.9 ± 0.1 |
| | 75 min | injection | | |
| | | 1 - 15 min | | |
| | Post-infusion | Post-drug | 11.9 ± 0.0 | 12.0 ± 0.0 |
| | 90 min | injection | | |
| | | 1 - 30 min | | |
| | Post-infusion | Post-drug | 11.9 ± 0.0 | 11.8 ± 0.1 |
| | 105 min | injection | | |
| | | 2 - 15 min | | |
| | Post-infusion | Post-drug | 11.9 ± 0.0 | 11.9 ± 0.1 |
| | 120 min | injection | | |
| | | 2 - 30 min | | |
| Delta changes | Post-infusion | | 95.7 ± 1.5* | 95.0 ± 0.9* |
| from Pre, sec | 15 min | | | |
| | Post-infusion | | 99.2 ± 0.8* | 100 ± 0* |
| | 30 min | | | |
| | Post-infusion | | 99.4 ± 0.8* | 99.1 ± 0.8* |
| | 45 min | | | |
| | Post-infusion | | 99.5 ± 0.8* | 99.0 ± 0.7* |
| | 60 min | | | |
| | Post-infusion | Post-drug | 100 ± 0* | 99.3 ± 0.7* |
| | 75 min | injection | | |
| | | 1 - 15 min | | |
| | Post-infusion | Post-drug | 98.7 ± 0.9* | 100 ± 0* |
| | 90 min | injection | | |
| | | 1 - 30 min | | |
| | Post-infusion | Post-drug | 99.2 ± 0.8* | 98.4 ± 1.1* |
| | 105 min | injection | | |
| | | 2 - 15 min | | |
| | Post-infusion | Post-drug | 99.3 ± 0.7* | 99.2 ± 0.8* |
| | 120 min | injection | | |
| | | 2 - 30 min | | |

### Righting Reflex

Prior to starting the fentanyl infusions (1 μg/kg/min, IV), it was essentially impossible to determine the righting reflex because the rats refused to be put on their side. Upon commencement of the infusion, the rats were obviously sedated and it was easy to place them on their side and at 5 min of infusion the two groups of rats (group 1 ultimately received injections of vehicle, group 2 ultimately received injections of L-NAC) got back on all four paws at 4.2±1.8 sec and 4.6±1.5 sec, respectively. From 15 min onward, none of the fentanyl-infused rats got back on their paws after being placed on their side (30 sec cut-off testing time) regardless of whether they ultimately received injections of vehicle or L-NAC.

This example shows that continuous intravenous infusion of fentanyl at 1 g/kg/min elicited pronounced sedative and antinociceptive effects in unanesthetized male Sprague-Dawley rats that were not affected by bolus injections of L-NAC (2×500 mol/kg, IV) that were given 60 min and 90 min time-points during infusion. A such, it is evident that these doses of L-NAC do not sufficiently rise to levels that may directly block opioid receptors or the signaling pathways that mediate these actions of fentanyl. In contrast, the findings that injections of L-NAC elicited a pronounced reversal of the adverse effects of fentanyl infusion of ventilatory parameters, ABG chemistry and A-a gradient provide direct evidence that L-NAC interferes with opioid-receptor signaling pathways that elicit the depression of breathing and alveolar gas exchange during continuous exposure to fentanyl. Our opinion would be that L-NAC ameliorates these effects by its propensity to alter the redox status or activities of intracellular signaling proteins rather than direct blockade of opioid receptors for which there is no direct evidence either way. Of vital interest to us is that the administration of bolus injections of L-NAC either immediately before or immediately after administrations of bolus injections of fentanyl or morphine were completely without effect. As such we are surprised at the efficacy of L-NAC in rats receiving the infusion of fentanyl and suspect that this means that the infusion causes intracellular signaling events including protein complexes that are susceptible to L-NAC or downstream products, but which are not sustained by bolus injections of opioids which are and therefore not affected by bolus injections of L-NAC given minutes afterward.

This example demonstrates that the infusion of fentanyl elicited pronounced changes in ventilatory parameters in unanesthetized rats. First, the fentanyl infusion elicited substantial and sustained decreases in Freq and TV and therefore pronounced reductions in MV. The fentanyl-induced decrease in Freq was associated with sustained increase in Ti whereas Te, which was decreased in the early stages of infusion, gradually reverted to a sustained increase in Te. EIP rose rapidly upon commencement of the fentanyl infusion whereas EEP abruptly began to rise at about 55 min of infusion. We do not understand any of the reasons for or mechanisms behind this pattern of responses, but it is obvious that fentanyl differentially affected the signaling pathways that control inspiration compared to those controlling expiration. Similarly, the infusion of fentanyl elicited a substantial and sustained decrease in PIF, and whereas it minimally affected PEF, it did increase EF₅₀ substantially, thereby promoting this aspect of expiratory mechanics. In addition, whereas fentanyl infusion decreased RT (reduced the time to expire 64% of TV) it greatly lengthened the difference in time between the decay of RT and Te, as expressed by greater Te-RT and apneic pause [(RT/Te)−1] values. As would be expected from the changes in TV, Ti and Te, the infusion of fentanyl elicited pronounced decreases in inspiratory drive (TV/Ti) and expiratory drive (TV/Te) most likely because of actions within the brainstem. We have reported that bolus injections of fentanyl (75 μg/kg, IV) markedly increase non-eupneic breathing events (NEBI) in unanesthetized rats due primarily to enhancing the incidence of apneas (see 10). In contrast, it is evident that the infusion of fentanyl had relatively minimal effects on NEBI. We assume that the levels of fentanyl and potential bioactive metabolites that are reached during the infusion do not reach those reached upon the bolus injection of the 75 μg/kg dose of fentanyl and therefore do not affect central and/or peripheral structures underlying the negative effects of the synthetic opioid on NEBI. The changes in ABG chemistry elicited by the infusion of fentanyl are consistent with hypoventilation whereas the increased A-a gradient is also consistent with the opioid causing a decrease in alveolar-gas exchange by mechanisms that likely involve atelectasis.

Key findings of described herein include that the bolus injections of L-NAC elicited a rapid and sustained reversal of many of the adverse effects of the fentanyl infusion on breathing. For example, the L-NAC injections reversed the fentanyl-induced depression of Freq, TV and MV with the enhancement of Freq associated with a profound reversal of the negative effects of fentanyl on Te and EEP but with minimal effects on the fentanyl-induced increase in Ti or EIP. As such it appears that L-NAC acts on the systems by which fentanyl infusion depresses TV and specifically affects the fentanyl-sensitive central/peripheral systems that regulate expiratory timing but not the fentanyl-sensitive systems that control inspiratory timing. As would be expected from the above findings, it was evident that L-NAC reversed the adverse effects of fentanyl infusion on both inspiratory and expiratory drives most likely by actions in brainstem sites expected to participate in the adverse effects of fentanyl on the drives to inhale and exhale. The findings that the injections of L-NAC reversed the negative effects of fentanyl infusion on PIF certainly suggests that L-NAC can reverse the deleterious effects of the fentanyl infusion on ventilatory mechanics associated with inspiration and perhaps those on the external intercostal muscles whereas the pronounced increases in PEF (despite minimal effects of fentanyl on this parameter) suggest that L-NAC can drive ventilatory mechanics associated with expiration including, perhaps, the activity of the internal intercostal muscles. In addition, although the injections of L-NAC did not influence the fentanyl-induced decrease in RT they did produce substantial reductions in the fentanyl-induced increases in Te-RT and apneic pause [(RT/Te)−1] values by specifically diminishing the fentanyl-induced increase in the duration of Te. Although the first injection of L-NAC reduced NEBI in the fentanyl-infused rats, it is difficult to interpret these data because of the relatively minor changes that are involved. Nonetheless, it could be said that L-NAC promotes somewhat the stability of eupneic breathing in the presence of the fentanyl infusion. Finally, the ability of L-NAC to rapidly overcome the adverse effects of the fentanyl infusion on arterial blood pH, pCO₂, pO₂ and sO₂ and A-gradient is consistent with the L-NAC-induced reversal of the adverse effects of the infusion on breathing and may speak to undefined actions of L-NAC within the lungs if the fentanyl-induced increase in A-a gradient is not simply due to hypoventilation-induced atelectasis.

In summary, this example shows that L-NAC can be employed to reverse OIRD in human subjects receiving a continuous intravenous infusion of fentanyl while maintaining the analgesic and sedative actions of the powerful synthetic opioid. L-NAC has been used in numerous clinical trial with various levels of success. Obviously, the efficacy of L-NAC upon oral or intravenous administration will depend upon numerous factors that affects its bioavailability (e.g., rate of degradation in the blood, liver and/or kidneys, formation of mixed disulfides) and rate of entry into cells.

TABLE 2
Definition of ventilatory parameters described in this study

| | | | |
|---|---|---|---|
| Parameter | Abbreviation | Units | Definition |

A. Directly recorded parameters

| | | | |
|---|---|---|---|
| Frequency of breaths | Freq | breaths/min | Rate of breathing |
| Inspiratory Time | Ti | sec | Duration of inspiration |
| Expiratory Time | Te | sec | Duration of expiration |
| End Inspiratory Pause | EIP | msec | Pause between end of inspiration |
| | | | start of expiration |
| End Expiratory Pause | EEP | msec | Pause between end of expiration |
| | | | and start of inspiration |
| Relaxation time | RT | sec | Decay of expiration to 36% |
| | | | maximum |
| Tidal Volume | TV | ml | Volume of inspired air per |
| | | | breath |
| Minute Ventilation | MV = freq × | ml/min | Total volume of air inspired per |
| | TV | | min |
| Peak Inspiratory Flow | PIF | ml/sec | Maximum inspiratory flow |
| Peak Expiratory Flow | PEF | ml/sec | Maximum expiratory flow |
| Expiratory flow at | EF₅₀ | ml/sec | Expiratory flow at 50% expired |
| 50% | | | TV |
| Rpef | Rpef | none | Time during expiration when |
| | | | peak expiratory flow occurs |
| Non-eupneic breathing | NEBI | % | % of non-eupneic breaths per |
| index | | | epoch |

B. Derived parameters

| | | | |
|---|---|---|---|
| Ti/Te | Ti/Te | none | Inspiratory quotient |
| Inspiratory Drive | TV/Ti | ml/sec | Central urge to inhale |
| Expiratory Drive | TV/Te | ml/sec | Central drive to exhale |
| PIF/PEF | PIF/PEF | none | Flow balance |
| NEBI/Frequency | NEBI/Freq | %/(b/min) | Rejection index corrected or |
| | | | Freq |
| Expiratory Time − | Te − RT | none | Relative expiratory delay |
| Relaxation Time | | | |
| Apneic Pause | (Te/RT) − 1 | none | Expiratory ratio |

TABLE 3
Baseline parameters prior to commencement of fentanyl infusion

| | Treatment Groups | |
|---|---|---|
| Parameter | Vehicle | L-NAC |
| Body Weights, gram | 326 ± 2 | 324 ± 2 |
| Frequency of Breathing (Freq), | 102 ± 3 | 102 ± 5 |
| breaths/min | | |
| Tidal Volume, mL | 2.41 ± 0.04 | 2.42 ± 0.05 |
| Minute Ventilation, mL/min | 246 ± 5 | 247 ± 14 |
| Inspiratory Time (Ti), sec | 0.184 ± 0.005 | 0.200 ± 0.010 |
| Expiratory Time (Te), sec | 0.425 ± 0.014 | 0.443 ± 0.022 |
| Ti/Te | 0.44 ± 0.02 | 0.46 ± 0.02 |
| End Inspiratory Pause, sec | 8.0 ± 0.5 | 7.9 ± 0.3 |
| End Expiratory Pause | 45.3 ± 1.6 | 44.3 ± 4.0 |
| Peak Inspiratory Flow (PIF), mL/sec | 17.7 ± 0.9 | 17.6 ± 1.1 |
| Peak Expiratory Flow (PEF), mL/sec | 11.3 ± 0.8 | 11.2 ± 0.7 |
| PIF/PEF | 1.60 ± 0.05 | 1.63 ± 0.11 |
| EF₅₀, mL/sec | 0.35 ± 0.03 | 0.36 ± 0.03 |
| Relaxation Time (RT), sec | 0.32 ± 0.01 | 0.32 ± 0.02 |
| Inspiratory Drive (TV/Ti), mL/sec | 13.1 ± 0.4 | 12.4 ± 0.6 |
| Expiratory Drive (TV/Te), mL/sec | 5.7 ± 0.2 | 5.5 ± 0.3 |
| Pause, (Te/RT) − 1 | 0.34 ± 0.06 | 0.40 ± 0.08 |
| Te − RT | 0.11 ± 0.02 | 0.12 ± 0.02 |
| Non-Eupneic Breathing Index, % | 2.53 ± 0.27 | 2.59 ± 0.42 |

L-NAC, N-acetyl-L-cysteine. The data are presented as mean ± SEM. There were 6 rats in each group. There were no between-group differences for any parameter (P > 0.05, for all between-group comparisons).

TABLE 4
Body temperatures during various stages of the experiments

| | | Injection | Study Group | |
|---|---|---|---|---|
| Parameters | Study Phase | Phase | Vehicle | L-NAC |
| Body | Pre | | 37.4 ± 0.1 | 37.4 ± 0.1 |
| temperature, ° C. | | | | |
| | Post infusion 15 min | | 37.5 ± 0.1 | 37.6 ± 0.1 |
| | Post infusion 30 min | | 37.6 ± 0.1 | 37.7 ± 0.1 |
| | Post infusion 45 min | | 37.7 ± 0.1 | 37.8 ± 0.1 |
| | Post infusion 60 min | | 37.8 ± 0.1 | 37.8 ± 0.1 |
| | Post-infusion 75 min | Post-drug | 37.8 ± 0.1 | 37.7 ± 0.1 |
| | | injection | | |
| | | 1 - 15 min | | |
| | Post-infusion 90 min | Post-drug | 37.7 ± 0.1 | 37.7 ± 0.1 |
| | | injection | | |
| | | 1 - 30 min | | |
| | Post-infusion 105 min | Post-drug | 37.8 ± 0.1 | 37.7 ± 0.1 |
| | | injection | | |
| | | 2 - 15 min | | |
| | Post-infusion 120 min | Post-drug | 37.8 ± 0.1 | 37.7 ± 0.1 |
| | | injection | | |
| | | 2 - 30 min | | |
| Delta changes | Post infusion 15 min | | 0.10 ± 0.04 | 0.14 ± 0.04* |
| from Pre, ° C. | | | | |
| | Post infusion 30 min | | 0.22 ± 0.03* | 0.23 ± 0.03* |
| | Post infusion 45 min | | 0.32 ± 0.04* | 0.33 ± 0.04* |
| | Post infusion 60 min | | 0.34 ± 0.05* | 0.34 ± 0.04* |
| | Post-infusion 75 min | Post-drug | 0.34 ± 0.06* | 0.28 ± 0.07* |
| | | injection | | |
| | | 1 - 15 min | | |
| | Post-infusion 90 min | Post-drug | 0.29 ± 0.07* | 0.32 ± 0.06* |
| | | injection | | |
| | | 1 - 30 min | | |
| | Post-infusion 105 min | Post-drug | 0.36 ± 0.07* | 0.28 ± 0.06* |
| | | injection | | |
| | | 2 - 15 min | | |
| | Post-infusion 120 min | Post-drug | 0.34 ± 0.05* | 0.29 ± 0.05* |
| | | injection | | |
| | | 2 - 30 min | | |

L-NAC, N-acetyl-L-cysteine (500 umol/kg, IV). The data are presented as mean ± SEM.
There were 9 rats in each group.
*P < 0.05, significant change from Pre-values. There were no between-group differences at any time-point (P > 0.05, for all comparisons).

TABLE 5
total arithmetic changes in body temperatures and
total percent changes in ventilatory parameters
during the first 60 min of fentanyl infusion

| | Treatment Groups | |
|---|---|---|
| Parameter | Vehicle | L-NAC |
| Frequency of Breathing (Freq), | −20.4 ± 1.9* | −21.6 ± 2.4* |
| breaths/min | | |
| Tidal Volume, mL | −22.7 ± 3.6* | −23.5 ± 2.5* |
| Minute Ventilation, mL/min | −38.2 ± 3.6* | −39.9 ± 2.7* |
| Inspiratory Time (Ti), sec | +94.2 ± 9.4* | +82.5 ± 14.1* |
| Expiratory Time (Te), sec | −5.1 ± 2.9* | −10.3 ± 3.5* |
| Ti/Te | +107.6 ± 12.3* | +104.4 ± 11.6* |
| End Inspiratory Pause, sec | +140.7 ± 17.2 | +147.0 ± 12.9 |
| End Expiratory Pause | +6.9 ± 8.1 | +9.1 ± 6.3 |
| Peak Inspiratory Flow (PIF), mL/sec | −44.5 ± 3.1* | −44.0 ± 3.7* |
| Peak Expiratory Flow (PEF), mL/sec | −2.7 ± 10.1 | −2.2 ± 2.8 |
| PIF/PEF | −41.3 ± 3.6* | −42.1 ± 4.7* |
| EF₅₀, mL/sec | +60.3 ± 12.5* | +57.6 ± 11.3* |
| Relaxation Time (RT), sec | −41.9 ± 1.9* | −41.4 ± 4.6* |
| Inspiratory Drive (TV/Ti), mL/sec | −57.7 ± 3.6* | −56.3 ± 2.7* |
| Expiratory Drive (TV/Te), mL/sec | −18.2 ± 4.5* | −14.6 ± 3.1* |
| Pause, (Te/RT) − 1 | +396.4 ± 62.9* | +348.6 ± 67.4* |
| Te − RT | +165.5 ± 30.5* | +129.9 ± 28.2* |
| Non-Eupneic Breathing Index | −39.8 ± 4.1* | −46.3 ± 3.0* |

L-NAC, N-acetyl-L-cysteine. The data are presented as mean ± SEM. There were 6 rats in each group. There were no between-group differences for any parameter (P > 0.05, for all between-group comparisons).

TABLE 6
ventilatory parameters and key stages of the experiment

| | | Actual values | | | | Arithmetic changes | | |
|---|---|---|---|---|---|---|---|---|
| Parameter | Group | Pre | 60 min | 90 min | 120 min | 60 min | 90 min | 120 min |
| Freq, | VEH | 102 ± | 82 ± | 75 ± | 71 ± | −20 ± | −27 ± | −31 ± |
| breaths/ | | 3 | 2 | 5 | 4 | 3* | 3* | 6* |
| min | | | | | | | | |
| | L-NAC | 102 ± | 78 ± | 90 ± | 89 ± | −24 ± | −13 ± | −13 ± |
| | | 5 | 5 | 4 | 4 | 5* | 3*^{,†} | 4*^{,†} |
| TV, mL | VEH | 2.41 ± | 1.65 ± | 1.74 ± | 1.85 ± | −0.76 ± | −0.67 ± | −0.56 ± |
| | | 0.04 | 0.13 | 0.14 | 0.13 | 0.13* | 0.13* | 0.12* |
| | L-NAC | 2.42 ± | 1.73 ± | 2.34 ± | 2.71 ± | −0.69 ± | −0.08 ± | +0.29 ± |
| | | 0.05 | 0.02 | 0.16 | 0.14 | 0.04* | 0.13^{†} | 0.13^{†} |
| MV, | VEH | 246 ± | 135 ± | 131 ± | 135 ± | −112 ± | 115 ± | −112 ± |
| mL/min | | 5 | 9 | 15 | 17 | 12* | 17* | 19* |
| | L-NAC | 247 ± | 135 ± | 210 ± | 241 ± | −111 ± | −37 ± | −6 ± |
| | | 14 | 10 | 20 | 14 | 10* | 13*^{,†} | 16^{†} |
| Ti, sec | VEH | 0.18 ± | 0.37 ± | 0.38 ± | 0.41 ± | +0.19 ± | +0.20 ± | 0.23 ± |
| | | 0.01 | 0.02 | 0.02 | 0.02 | 0.02 | 0.02* | 0.03* |
| | L-NAC | 0.20 ± | 0.36 ± | 0.36 ± | 0.35 ± | +0.16 ± | +0.15 ± | 0.15 ± |
| | | 0.01 | 0.02 | 0.01 | 0.02 | 0.03* | 0.02* | 0.02* |
| Te, sec | VEH | 0.43 ± | 0.42 ± | 0.48 ± | 0.52 ± | −0.01 ± | −0.05 ± | −0.10 ± |
| | | 0.02 | 0.03 | 0.02 | 0.04 | 0.04 | 0.03 | 0.04* |
| | L-NAC | 0.44 ± | 0.38 ± | 0.34 ± | 0.34 ± | −0.06 ± | −0.10 ± | −0.11 ± |
| | | 0.02 | 0.02 | 0.03 | 0.02 | 0.02 | 0.04* | 0.02* |
| Ti/Te | VEH | 0.44 ± | 0.91 ± | 0.81 ± | 0.80 ± | +0.47 ± | +0.37 ± | +0.37 ± |
| | | 0.02 | 0.06 | 0.03 | 0.05 | 0.08* | 0.05* | 0.06* |
| | L-NAC | 0.46 ± | 0.96 ± | 1.06 ± | 1.05 ± | +0.51 ± | +0.60 ± | +0.60 ± |
| | | 0.02 | 0.05 | 0.07 | 0.07 | 0.05* | 0.06*^{,†} | 0.06*^{,†} |
| EIP, | VEH | 8.0 ± | 20.0 ± | 18.7 ± | 21.8 ± | +12 ± | +11 ± | +14 ± |
| msec | | 0.5 | 0.8 | 1.1 | 1.7 | 1* | 2* | 2* |
| | L-NAC | 7.9 ± | 20.2 ± | 20.2 ± | 19.7 ± | +12 ± | +12 ± | +12 ± |
| | | 0.3 | 1.5 | 1.6 | 1.1 | 2* | 2* | 1* |
| EEP, | VEH | 45.3 ± | 79.8 ± | 132.7 ± | 123.3 ± | +35 ± | +87 ± | +78 ± |
| msec | | 1.6 | 7.6 | 10.2 | 9.2 | 9* | 11* | 10* |
| | L-NAC | 45.4 ± | 79.7 ± | 16.1 ± | 14.2 ± | +34 ± | −30 ± | −31 ± |
| | | 4.1 | 4.2 | 1.5 | 2.4 | 7* | 4*^{,†} | 3*^{,†} |
| PIF, mL | VEH | 17.7 ± | 8.5 ± | 8.7 ± | 8.3 ± | −9.2 ± | −8.9 ± | −9.4 ± |
| | | 0.9 | 0.6 | 1.1 | 0.8 | 1.2* | 1.8* | 1.4* |
| | L-NAC | 17.6 ± | 9.0 ± | 11.4 ± | 14.2 ± | −8.7 ± | −6.3 ± | −3.5 ± |
| | | 1.1 | 0.6 | 0.7 | 0.5 | 1.4* | 0.9* | 0.1* |
| PEF | VEH | 11.3 ± | 10.6 ± | 10.5 ± | 11.3 ± | −0.63 ± | −0.75 ± | +0.05 ± |
| | | 0.8 | 0.6 | 0.9 | 1.1 | 0.83 | 0.30 | 1.01 |
| | L-NAC | 11.2 ± | 11.2 ± | 13.8 ± | 15.3 ± | −0.06 ± | +2.53 ± | +4.01 ± |
| | | 0.7 | 0.6 | 1.0 | 0.7 | 1.18 | 0.66* | 1.18*^{,†} |
| PIF/PEF | VEH | 1.60 ± | 0.80 ± | 0.86 ± | 0.74 ± | −0.80 ± | −0.74 ± | −0.86 ± |
| | | 0.12 | 0.04 | 0.13 | 0.05 | 0.14* | 0.17* | 0.10* |
| | L-NAC | 1.61 ± | 0.82 ± | 0.84 ± | 0.93 ± | −0.78 ± | −0.76 ± | −0.67 ± |
| | | 0.11 | 0.08 | 0.05 | 0.03 | 0.16* | 0.09* | 0.13* |

VEH, vehicle.
L-NAC, N-acetyl-L-cysteine.
Freq, frequency of breathing, TV, tidal volume.
MV, minute ventilation.
Ti, inspiratory time.
Te, expiratory time.
EIP, end inspiratory pause.
EEP, end expiratory pause.
PIF, peak inspiratory flow.
PEF, peak expiratory flow.
Data are shown as mean ± SEM. There were 6 rats in each group.
*P < 0.05, significant change from Pre values.
^{†}P < 0.05, L-NAC versus VEH.

TABLE 8
Ventilatory parameters and key stages of the experiment

| | | Actual values | | | | Arithmetic changes | | |
|---|---|---|---|---|---|---|---|---|
| Parameter | Group | Pre | 60 min | 90 min | 120 min | 60 min | 90 min | 120 min |
| EF₅₀, mL | VEH | 0.35 ± | 0.57 ± | 0.55 ± | 0.35 ± | +0.22 ± | +0.20 ± | +0.24 ± |
| | | 0.03 | 0.02 | 0.04 | 0.03 | 0.04* | 0.04* | 0.07* |
| | L-NAC | 0.36 ± | 0.63 ± | 0.60 ± | 0.64 ± | +0.27 ± | +0.24 ± | +0.28 ± |
| | | 0.03 | 0.08 | 0.07 | 0.05 | 0.06* | 0.08* | 0.05* |
| RT, sec | VEH | 0.32 ± | 0.17 ± | 0.17 ± | 0.16 ± | −0.15 ± | −0.15 ± | −0.15 ± |
| | | 0.01 | 0.01 | 0.02 | 0.01 | 0.01* | 0.02* | 0.02* |
| | L-NAC | 0.32 ± | 0.17 ± | 0.18 ± | 0.18 ± | −0.15 ± | −0.14 ± | −0.14 ± |
| | | 0.02 | 0.02 | 0.01 | 0.02 | 0.02* | 0.02* | 0.02* |
| InspD | VEH | 13.1 ± | 4.5 ± | 4.6 ± | 4.7 ± | −8.7 ± | −8.6 ± | −8.5 ± |
| (TV/Ti), | | 0.4 | 0.4 | 0.4 | 0.5 | 0.6* | 0.6* | 0.8* |
| mL/sec | | | | | | | | |
| | L-NAC | 12.4 ± | 4.8 ± | 6.6 ± | 7.9 ± | −7.5 ± | −5.6 ± | −4.5 ± |
| | | 0.6 | 0.3 | 0.4 | 0.6 | 0.7* | 0.7*^{,†} | 0.6*^{,†} |
| ExpD | VEH | 5.7 ± | 4.1 ± | 3.7 ± | 3.7 ± | −1.7 ± | −2.1 ± | −2.1 ± |
| TV/Te, | | 0.2 | 0.4 | 0.3 | 0.4 | 0.5* | 0.4* | 0.4* |
| mL/sec | | | | | | | | |
| | L-NAC | 5.5 ± | 4.6 ± | 6.9 ± | 8.1 ± | −0.9 ± | +1.4 ± | +2.6 ± |
| | | 0.3 | 0.2 | 0.4 | 0.4 | 0.2* | 0.6^{†} | 0.4*^{,†} |
| Pause, | VEH | 0.34 ± | 1.51 ± | 1.95 ± | 2.23 ± | +1.17 ± | +1.61 ± | +1.89 ± |
| (Te/RT)-1 | | 0.06 | 0.24 | 0.28 | 0.35 | 0.22* | 0.23* | 0.29* |
| | L-NAC | 0.40 ± | 1.32 ± | 0.94 ± | 0.94 ± | +0.92 ± | +0.54 ± | 0.53 ± |
| | | 0.08 | 0.16 | 0.13 | 0.14 | 0.16* | 0.14*^{,†} | 0.09*^{,†} |
| Te-RT | VEH | 0.11 ± | 0.25 ± | 0.31 ± | 0.36 ± | +0.14 ± | +0.20 ± | +0.25 ± |
| | | 0.02 | 0.04 | 0.02 | 0.05 | 0.04* | 0.02* | 0.03* |
| | L-NAC | 0.12 ± | 0.21 ± | 0.17 ± | 0.16 ± | +0.09 ± | +0.04 ± | +0.04 ± |
| | | 0.02 | 0.02 | 0.02 | 0.02 | 0.02* | 0.03^{†} | 0.02^{†} |
| NEBI, % | VEH | 2.5 ± | 2.8 ± | 2.5 ± | 1.2 ± | +0.3 ± | −0.0 ± | −1.3 ± |
| | | 0.3 | 0.8 | 0.8 | 0.6 | 0.7 | 0.7 | 0.6* |
| | L-NAC | 2.6 ± | 2.0 ± | 1.0 ± | 1.3 ± | −0.6 ± | −1.6 ± | −1.3 ± |
| | | 0.4 | 0.7 | 0.7 | 0.7 | 0.6 | 0.9 | 0.9* |

VEH, vehicle.
L-NAC, N-acetyl-L-cysteine.
EF₅₀, expiratory flow at 50% expired tidal volume.
RT, relaxation time.
TV, tidal volume.
Ti, inspiratory time.
Te, expiratory time.
InspD, inspiratory drive (TV/Ti).
ExpD, expiratory drive (TV/Te).
RT, relaxation time.
NEBI, non-eupneic breathing index.
Data are shown as mean ± SEM. There were 6 rats in each group.
*P < 0.05, significant change from Pre values.
^{†}P < 0.05, L-NAC versus VEH.

TABLE 9
Arterial blood gas chemistry values during key stages of the experiment

| | | | | 30 min post- | 30 min post- |
|---|---|---|---|---|---|
| | | | | injection 1 | injection 2 |
| | | | Time of fentanyl infusion (min) | | |
| Parameter | | Pre | 60 | 90 | 120 |
| pH | Vehicle | 7.425 ± 0.009 | 7.180 ± 0.038 | 7.168 ± 0.033 | 7.157 ± 0.030 |
| | L-NAC | 7.442 ± 0.008 | 7.162 ± 0.026 | 7.392 ± 0.006 | 7.443 ± 0.013 |
| pCO₂, mmHg | Vehicle | 35.8 ± 0.05 | 59.1 ± 1.6 | 59.7 ± 1.5 | 60.6 ± 1.6 |
| | L-NAC | 35.7 ± 0.03 | 57.3 ± 1.5 | 36.2 ± 1.1 | 35.2 ± 0.4 |
| pO₂, mmHg | Vehicle | 92.4 ± 0.06 | 58.3 ± 1.3 | 55.3 ± 1.4 | 50.0 ± 1.7 |
| | L-NAC | 92.1 ± 0.07 | 58.4 ± 1.9 | 86.3 ± 1.6 | 84.5 ± 1.4 |
| sO₂, % | Vehicle | 98.2 ± 0.7 | 58.7 ± 1.1 | 58.9 ± 1.2 | 55.9 ± 2.7 |
| | L-NAC | 99.0 ± 0.4 | 59.1 ± 1.8 | 93.6 ± 0.9 | 92.4 ± 1.2 |
| A-a gradient, | Vehicle | 17.6 ± 0.8 | 25.7 ± 1.7 | 28.1 ± 1.4 | 32.4 ± 2.3 |
| mmHg | L-NAC | 17.9 ± 0.7 | 27.6 ± 0.8 | 23.1 ± 1.3 | 26.1 ± 1.5 |

L-NAC, N-acetyl-L-cysteine (500 μmol/kg, IV). A-a gradient, Alveolar-arterial gradient. The data are presented as mean ± SEM. There were 6 rats in each group.

TABLE 10
Ventilatory responses elicited by morphine and subsequent injections
of vehicle or N-acetyl-L-cysteine dimethyl ester (L-NACme)

| | | Treatment Groups | |
|---|---|---|---|
| Parameters | Stage of Experiment | Vehicle | L-NACme |
| Number | Morning of experiment | 9 | 9 |
| Age, days | Morning of experiment | 80.0 ± 0.5 | 79.7 ± 0.4 |
| Body weights, g | Morning of experiment | 337 ± 3 | 336 ± 2 |
| Frequency | Pre values | 99 ± 3 | 98 ± 4 |
| | Morphine - peak (+) response (%) | +47 ± 6* | +42 ± 6* |
| | Morphine peak (−) response (%) | −24 ± 3* | −22 ± 4* |
| | Morphine - first 15 min response (%) | −4.0 ± 2.1 | −4.9 ± 2.8 |
| | Drug - entire 75 min, % change | +18 ± 4* | +11 ± 2* |
| Tidal Volume | Pre values | 2.62 ± 0.13 | 2.61 ± 0.16 |
| | Morphine - peak (+) response (%) | −19 ± 3* | −16 ± 6* |
| | Morphine peak (−) response (%) | −38 ± 5* | −40 ± 6* |
| | Morphine - first 15 min response (%) | −33 ± 4* | −34 ± 3* |
| | Drug - entire 75 min, % change | −23 ± 4* | −22 ± 3* |
| Minute Ventilation | Pre values | 256 ± 6 | 259 ± 17 |
| | Morphine - peak (+) response (%) | +19 ± 5* | +20 ± 6* |
| | Morphine peak (−) response (%) | −53 ± 5* | −54 ± 4* |
| | Morphine - first 15 min response (%) | −36 ± 3* | −35 ± 3* |
| | Drug - entire 75 min, % change | −15 ± 2* | −19 ± 4* |

Drug refers to two injections of vehicle or N-acetyl-L-cysteine ethyl ester (L-NACme; 500 μmol/kg, IV). The data are presented as mean ± SEM. There were 9 rats in each group. There were no between group differences for any Pre-value (P > 0.05, for all comparisons.
*P < 0.05, significant response.
^{†}P < 0.05, value in the D-cystine group versus value in the vehicle group.

### Example 2

### Bolus Injections of L-NAC Overcome Fentanyl-Induced Constipation and Anuria

We performed experiments to determine whether bolus injections of N-acetyl-L-cysteine (L-NAC) could overcome the constipation and anuria (loss of urine excretion) elicited by the infusion of fentanyl in rats.

### Methods

Adult male Sprague-Dawley rats were placed in placed in individual metabolic cages that allowed pellets and urine to drop into a receptacle (pellets that did not drop to the receptacle were manually removed). The rats received infusions of vehicle or fentanyl via an indwelling jugular catheter for 6 h starting at 6.30 PM, 30 min after the start of the dark cycle, when rats are most active and secreting a substantial percentage of daily fecal and urine output. Pellets were constantly removed by specialized tongs and urine was constantly removed by pipette. Bolus injections of vehicle or L-NAC were given by another indwelling jugular vein catheter.

Treatment Groups (n=9 Rats Per Group)
Group 1: Vehicle infusion (100 μL/h for 6 h) plus injections of vehicle after 60 and 90 min.
Group 2: Fentanyl infusion (1 μg/kg/min for 6 h, 100 L/h), injections of vehicle after 60 and 90 min.
Group 3: Vehicle infusion (100 μL/hour for 6 h) plus injections of L-NAC (500 mol/kg) at 60 and 90 min.
Group 4: Fentanyl infusion (1 μg/kg/min for 6 h, 100 L/h), injections of L-NAC (500 mol/kg) at 60 and 90 min.

### Sampling Time

Urine and fecal samples taken over the 5 h following the bolus injections of vehicle or L-NAC.

### Results

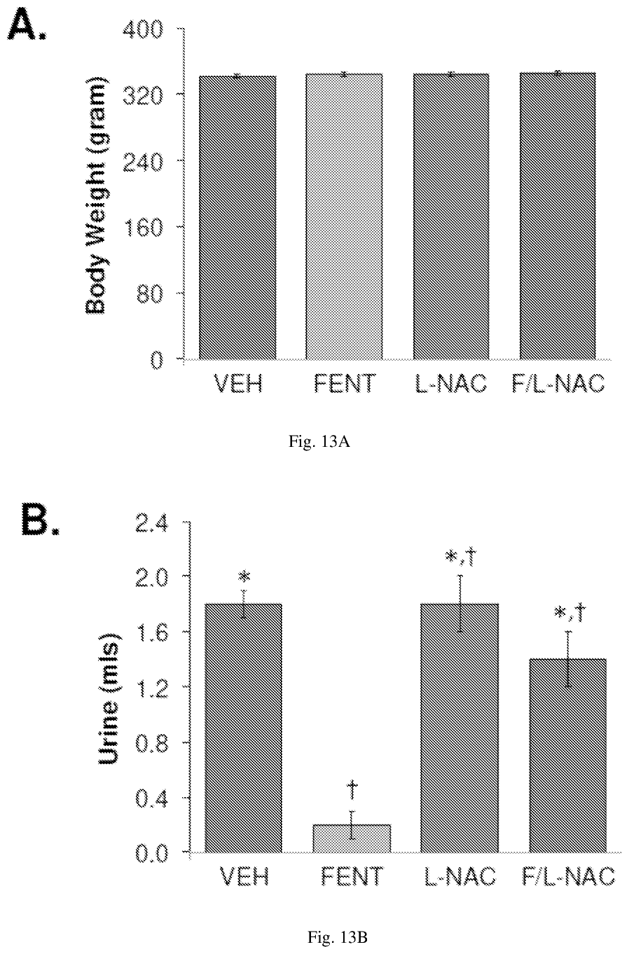

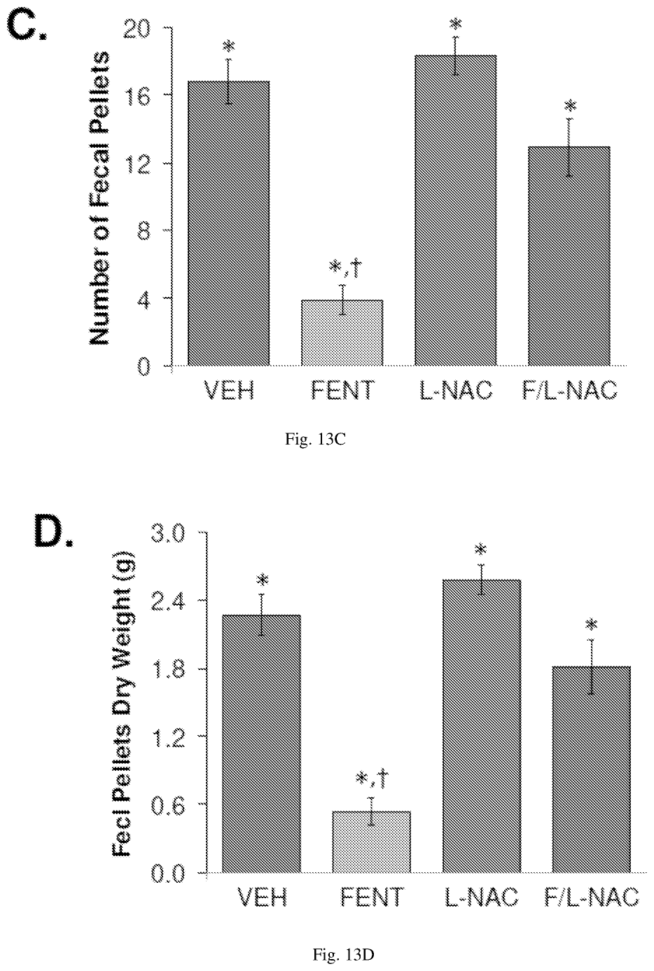

FIGS. 13(A-D) show bolus injections of L-NAC did not affect parameters in vehicle-infused rats. Panel A: The body weights of the four groups were similar to one another. Panel B: Fentanyl infusion markedly decreased urine production (anuria); which was normalized by L-NAC injections (F/L-NAC). Panels C and D: Fentanyl infusion markedly decreased fecal output (number of pellets, total weight), which was normalized by L-NAC injections.

The bolus administration of L-NAC at 60 and 90 min of fentanyl infusion dramatically impacts the deleterious effects of fentanyl on fecal and urine production over 5 hours of infusion.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A pharmaceutical composition comprising:
a compound having a structure of formulas: 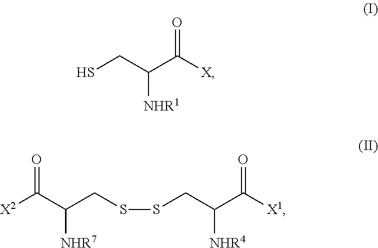
or an adduct, a pharmaceutically acceptable salt, a tautomer, or a solvate thereof; wherein:
X is OR² or N(R³)₂;
X¹ is OR⁵ or N(R⁶)₂;
X² is OR⁸ or N(R⁹)₂;
R¹, R⁴, and R⁷ are each independently H, alkyl optionally substituted with one or more halogen, or —C(O)-alkyl optionally substituted with one or more halogen;
R², R⁵, and R⁸ are each independently H or alkyl optionally substituted with one or more halogen;
each R³, R⁶, and R⁹ are independently H or alkyl optionally substituted with one or more halogen;
X is not OR² if R¹ is H; and
X² is not OR⁸ if X¹ is OR⁵ and R⁴ and R⁷ are H.

2: The composition of claim 1, wherein the adduct of the compound of formulas I or II is biologically active and includes at least one of an albumin adduct, a glucose adduct, an L-cysteine adduct, an L-glutathione adduct, an D-cysteine adduct, or an S-nitroso adduct.

3: The composition of claim 1, wherein R¹, R⁴, and R⁷ are each independently H, C₁-C₆ alkyl optionally substituted with one or more halogen, or —C(O)—(C₁-C₆ alkyl) optionally substituted with one or more halogen.

4: The composition of claim 1, wherein R¹, R⁴, and R⁷ are each independently H, methyl, ethyl, propyl, butyl, —C(O)— methyl, —C(O)-ethyl, —C(O)-propyl, or —C(O)-butyl, each optionally substituted with one or more halogen.

5: The composition of claim 1, wherein R², R⁵, and R⁸ are each independently H or C₁-C₆ alkyl optionally substituted with one or more halogen.

6: The composition of claim 1, wherein R², R⁵, and R⁸ are each independently H, methyl, ethyl, or propyl, or butyl, each optionally substituted with one or more halogen.

7: The composition of claim 1, wherein each R³, R⁶, and R⁹ is independently H or C₁-C₆ alkyl optionally substituted with one or more halogen.

8: The composition of claim 1, wherein one of each R³, R⁶, and R⁹ is H and the other of R³, R⁶, and R⁹ is methyl, ethyl, or propyl, or butyl, each optionally substituted with one or more halogen.

9: The composition of claim 1, wherein X is OR², R¹ is H, C₁-C₆ alkyl optionally substituted with one or more halogen, or —C(O)—(C₁-C₆ alkyl) optionally substituted with one or more halogen, and R² is H or C₁-C₆ alkyl optionally substituted with one or more halogen.

10: The composition of claim 1, wherein X is OR², R¹ is H, methyl, ethyl, propyl, butyl, —C(O)-methyl, —C(O)-ethyl, —C(O)-propyl, or —C(O)-butyl, each optionally substituted with one or more halogen, and R² is H, methyl, ethyl, or propyl, or butyl, each optionally substituted with one or more halogen.

11: The composition of claim 1, wherein X is N(R³)₂; R¹ is H, C₁-C₆ alkyl optionally substituted with one or more halogen, or —C(O)—(C₁-C₆ alkyl) optionally substituted with one or more halogen; and each R³ is independently H or C₁-C₆ alkyl optionally substituted with one or more halogen.

12: The composition of claim 1, wherein X is N(R³)₂, R¹ is H, methyl, ethyl, propyl, butyl, —C(O)-methyl, —C(O)-ethyl, —C(O)-propyl, or —C(O)-butyl, each optionally substituted with one or more halogen, and one R³ is H and the other R³ is methyl, ethyl, or propyl, or butyl, each optionally substituted with one or more halogen.

13: The composition of claim 1, wherein X¹ is OR⁵, R⁴ is H, C₁-C₆ alkyl optionally substituted with one or more halogen, or —C(O)—(C₁-C₆ alkyl) optionally substituted with one or more halogen, R⁵ is H or C₁-C₆ alkyl optionally substituted with one or more halogen; X² is OR⁸, R⁷ is H, C₁-C₆ alkyl optionally substituted with one or more halogen, or —C(O)—(C₁-C₆ alkyl) optionally substituted with one or more halogen, and R⁸ is H or C₁-C₆ alkyl optionally substituted with one or more halogen.

14: The composition of claim 1, wherein X¹ is OR⁵, R⁴ is H, methyl, ethyl, propyl, butyl, —C(O)-methyl, —C(O)-ethyl, —C(O)-propyl, or —C(O)-butyl, each optionally substituted with one or more halogen, R⁵ is H, methyl, ethyl, or propyl, or butyl, each optionally substituted with one or more halogen, X² is OR⁸; R⁷ is H, methyl, ethyl, propyl, butyl, —C(O)-methyl, —C(O)-ethyl, —C(O)-propyl, or —C(O)-butyl, each optionally substituted with one or more halogen, and R⁸ is H, methyl, ethyl, or propyl, or butyl, each optionally substituted with one or more halogen.

15: The composition of claim 1, wherein X¹ is N(R⁶)₂, R⁴ is H, C₁-C₆ alkyl optionally substituted with one or more halogen, or —C(O)—(C₁-C₆ alkyl) optionally substituted with one or more halogen, each R⁶ is independently H or C₁-C₆ alkyl optionally substituted with one or more halogen, X² is N(R⁹)₂, R⁷ is H, C₁-C₆ alkyl optionally substituted with one or more halogen, or —C(O)—(C₁-C₆ alkyl) optionally substituted with one or more halogen, and R⁹ is H or C₁-C₆ alkyl optionally substituted with one or more halogen.

16: The composition of claim 1, wherein X¹ is N(R³)₂, R⁴ is H, methyl, ethyl, propyl, butyl, —C(O)-methyl, —C(O)-ethyl, —C(O)-propyl, or —C(O)-butyl, each optionally substituted with one or more halogen, one R⁶ is H and the other R⁶ is methyl, ethyl, or propyl, or butyl, each optionally substituted with one or more halogen, X¹ is N(R⁹)₂; R⁷ is H, methyl, ethyl, propyl, butyl, —C(O)-methyl, —C(O)-ethyl, —C(O)-propyl, or —C(O)-butyl, each optionally substituted with one or more halogen, and one R⁹ is H and the other R⁹ is methyl, ethyl, or propyl, or butyl, each optionally substituted with one or more halogen.

17: The composition of claim 1, wherein the compound is not cysteine, cystine, a cysteine alkylester, or cystine dialkylester.

18-71. (canceled)

72: The composition of claim 1, further comprising a pharmaceutically acceptable carrier or excipient.

73: A method of attenuating opioid induced ventilatory and/or respiratory depression in a subject in need thereof, the method comprising:
administering to the subject a therapeutically effective amount of a composition of claim 1.

74-89. (canceled)
